SEQUENCE LISTING (13 sequences)

SEQ ID NO: 1 - protein, 5 aa, Artificial Sequence
  Repitition sequence of silk protein
  Xaa (4)..(5): each Xaa can be independently from another any naturally occurring amino acid
GPGXX

SEQ ID NO: 2 - protein, 5 aa, Artificial Sequence
  Repitition sequence of silk protein
  Xaa (5)..(5): Xaa can be any naturally occurring amino acid
GVPGX

SEQ ID NO: 3 - protein, 5 aa, Artificial Sequence
  Repitition sequence of silk protein
  Xaa (4)..(5): each Xaa can be independently from another any naturally occurring amino acid
GSGXX

SEQ ID NO: 4 - protein, 5 aa, Artificial Sequence
  Repitition sequence of silk protein
  Xaa (4)..(5): each Xaa can be independently from another any naturally occurring amino acid
GGYXX

SEQ ID NO: 5 - protein, 5 aa, Artificial Sequence
  Repitition sequence of silk protein
  Xaa (4)..(5): each Xaa can be independently from another any naturally occurring amino acid
PQQXX

SEQ ID NO: 6 - protein, 5 aa, Artificial Sequence
  Repitition sequence of silk protein
  Xaa (4)..(5): each Xaa can be independently from another any naturally occurring amino acid
GYGXX

SEQ ID NO: 7 - protein, 5263 aa, Bombyx mori
MRVKTFVILCCALQYVAYTNANINDFDEDYFGSDVTVQSSNTTDEIIRDASGAVIEEQITTKKMQRKNKNHGILGKNEKMIKTFVITTDSDGNESIVEEDVLMKTLSDGTVAQSYVAADAGAYSQSGPYVSNSGYSTHQGYTSDFSTSAAVGAGAGAGAAAGSGAGAGAGYGAASGAGAGAGAGAGAGYGTGAGAGAGAGYGAGAGAGAGAGYGAGAGAGAGAGYGAGAGAGAGAGYGAGAGAGAGAGYGAGAGAGAGAGYGAASGAGAGAGYGQGVGSGAASGAGAGAGAGSAAGSGAGAGAGTGAGAGYGAGAGAGAGAGYGAASGTGAGYGAGAGAGYGGASGAGAGAGAGAGAGAGAGYGTGAGYGAGAGAGAGAGAGAGYGAGAGAGYGAGYGVGAGAGYGAGYGAGAGSGAASGAGSGAGAGSGAGAGSGAGAGSGAGAGSGAGAGSGAGAGSGAGAGSGAGAGSGTGAGSGAGAGYGAGAGAGYGAGAGSGAASGAGAGSGAGAGSGAGAGSGAGAGSGAGAGSGAGAGYGAGAGAGYGAGAGAGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSRSDGYEYAWSSDFGTGSGAGAGSGAGAGSGAGAGSGAGAGSGAGAGSGAGAGYGAGVGVGYGAGYGAGAGAGYGAGAGSGAASGAGAGSGAGAGSGAGAGSGAGAGSGAGAGSGAGAGSGAGAGSGAGAGSGAGAGSGAGAGSGAGVGSGAGAGSGAGAGVGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGSGAGAGSGAGAGSGAGAGSGAGAGSGAGAGSGAGAGSGAGAGSGAGAGSGAGAGSGAGAGSGAGAGYGAGAGAGYGAGYGAGAGAGYGAGAGSGAASGAGSGAGAGSGAGAGAGSGAGAGSGAGAGSGAGAGSGAGAGSGAGAGSGAGAGYGAGVGAGYGAGYGAGAGAGYGAGAGSGAASGAGAGSGAGAGSGAGAGSGAGAGSGAGAGSGAGAGSGAGAGSGAGVGYGAGYGAGAGAGYGAGAGSGAASGAGAGAGAGAGTGSSGFGPYVAHGGYSGYEYAWSSESDFGTGSGAGAGSGAGAGSGAGAGSGAGAGSGAGYGAGVGAGYGAGYGAGAGAGYGAGAGSGAGSGAGAGSGAGAGSGAGAGSGAGAGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGAGYGAGVGAGYGAGYGAGAGAGYGAGAGSGAGSGAGAGSGAGAGSGAGAGSGAGVGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSGYEYAWSSESDFGTGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGSGSGAGAGSGAGAGSGAGAGYGAGVGAGYGVGYGAGAGAGYGAGAGSGAASGAGAGAGAGAGTGSSGFGPYVAHGGYSGYEYAWSSESDFGTGSGAGAGSGAGAGSGAGAGSGAGAGSGAGAGSGAGAGSGAGAGYGAGVGAGYGAAYGAGAGAGYGAGAGSGAASGAGAGSGAGAGSGAGAGSGAGAGSGAGAGSGAGAGSGAGAGSGAGAGSGAGAGSGAGAGSGAGAGYGAGAGAGYGAGAGSGAGSGAGAGSGAGAGSGAGAGSGAGAGSGAGAGSGAGSGSGAGAGSGAGAGSGAGAGYGAGVGAGYGAGYGAGAGAGYGAGAGSGAGSGAGAGSGAGAGYGAGAGAGYGAGYGAGAGAGYGAGAGTGAGSGAGAGSGAGAGSGAGAGSGAGAGSGAGAGSGAGAGSGAGSGSGAGAGSGAGAGSGAGAGSGAGAGSGAGAGSGAGAGYGAGAGAGYGAGYGAGAGAGYGAGAGSGAGSGAGAGSGAGAGSGAGAGSGAGAGYGAGYGAGAGSGAASGAGAGAGAGAGTGSSGFGPYVAHGGYSGYEYAWSSESDFGTGSGAGAGSGAGAGAGAGAGSGAGAGYGAGVGAGYGAGYGAGAGAGYGAGAGSGTGSGAGAGSGAGAGYGAGVGAGYGAGAGSGAAFGAGAGAGAGSGAGAGSGAGAGSGAGAGSGAGAGSGAGAGYGAGYGAGVGAGYGAGAGSGAASGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGSGAGAGSGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGYGAGAGSGAASGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGSGAGSGAGAGSGAGAGSGAGAGSGAGAGYGAGVGAGYGVGYGAGVGAGYGAGAGSGAASGAGAGSGAGAGAGSGAGAGSGAGAGSGAGAGSGAGAGSGAGAGSGAGAGYGAGYGAGVGAGYGAGAGVGYGAGAGAGYGAGAGSGAASGAGAGAGSGAGAGTGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGAGSGAGSGAGAGSGAGAGSGAGAGSGAGAGSGAGAGYGAGAGSGTGSGAGAGSGAGAGSGAGAGSGAGAGSGAGAGSGAGAGSGVGAGYGVGYGAGAGAGYGVGYGAGAGAGYGAGAGSGTGSGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGYGAGVGAGYGVGYGAGAGAGYGAGAGSGAASGAGAGAGAGAGTGSSGFGPYVANGGYSGYEYAWSSESDFGTGSGAGAGSGAGAGSGAGAGYGAGYGAGVGAGYGAGAGVGYGAGAGAGYGAGAGSGAASGAGAGAGAGAGSGAGAGSGAGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGYGAGYGAGVGAGYGAGAGVGYGAGAGAGYGAGAGSGAASGAGAGAGAGAGAGSGAGAGSGAGAGSGAGAGSGAGAGSGAGAGSGAGAGSGAGAGSGAGAGSGAGAGYGAGVGAGYGAGYGGAGAGYGAGAGSGAASGAGAGSGAGAGSGAGAGSGAGAGSGAGAGSGAGAGYGAGAGSGAASGAGAGAGAGAGTGSSGFGPYVNGGYSGYEYAWSSESDFGTGSGAGAGSGAGAGSGAGAGYGAGVGAGYGAGYGAGAGAGYGAGAGSGAASGAGAGSGAGAGSGAGAGSGAGAGSGAGSGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGAGSGAGAGSGAGSGAGAGSGAGAGYGAGYGAGVGAGYGAGAGVGYGAGAGAGYGAGAGSGAASGAGAGSGSGAGSGAGAGSGAGAGSGAGAGAGSGAGAGSGAGAGSGAGAGYGAGYGAGAGSGAASGAGAGAGAGAGTGSSGFGPYVANGGYSGYEYAWSSESDFGTGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGSGAGAGYGAGYGAGAGAGYGAGAGVGYGAGAGAGYGAGAGSGAGSGAGAGSGSGAGAGSGSGAGSGAGAGSGAGAGSGAGAGSGAGAGSGAGAGSGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGSGAGAGSGAGAGSGAGAGYGAGAGVGYGAGAGSGAASGAGAGSGAGAGSGAGAGSGAGAGSGAGAGSGAGAGSGAGAGSGAGSGAGAGSGAGAGYGAGYGAGVGAGYGAGAGYGAGYGVGAGAGYGAGAGSGAGSGAGAGSGAGAGSGAGAGSGAGAGSGAGAGSGAGSGAGAGYGAGAGAGYGAGAGAGYGAGAGSGAASGAGAGAGAGSGAGAGSGAGAGSGAGSGAGAGSGAGAGYGAGAGSGAASGAGAGSGAGAGAGAGAGAGSGAGAGSGAGAGYGAGAGSGAASGAGAGAGAGTGSSGFGPYVANGGYSRREGYEYAWSSKSDFETGSGAASGAGAGAGSGAGAGSGAGAGSGAGAGSGAGAGGSVSYGAGRGYGQGAGSAASSVSSASSRSYDYSRRNVRKNCGIPRRQLVVKFRALPCVNC

SEQ ID NO: 8 - protein, 262 aa, Bombyx mori
MKPIFLVLLVATSAYAAPSVTINQYSDNEIPRDIDDGKASSVISRAWDYVDDTDKSIAILNVQEILKDMASQGDYASQASAVAQTAGIIAHLSAGIPGDACAAANVINSYTDGVRSGNFAGFRQSLGPFFGHVGQNLNLINQLVINPGQLRYSVGPALGCAGGGRIYDFEAAWDAILASSDSSFLNEEYCIVKRLYNSRNSQSNNIAAYITAHLLPPVAQVFHQSAGSITDLLRGVGNGNDATGLVANAQRYIAQAASQVHV

SEQ ID NO: 9 - protein, 1186 aa, Bombyx mori
MRFVLCCTLIALAALSVKAFGHHPGNRDTVEVKNRKYNAASSESSYLNKDNDSISAGAHRAKSVEQSQDKSKYTSGPEGVSYSGRSQNYKDSKQAYADYHSDPNGGSASAGQSRDSSLRERNVHYVSDGEAVAASSDARDENRSAQQNAQANWNADGSYGVSADRSGSASSRRRQANYYSDKDITAASKDDSRADSSRRSNAYYNRDSDGSESAGLSDRSASSSKNDNVFVYRTKDSIGGQAKSSRSSHSQESDAYYNSSPDGSYNAGTRDSSISNKKKASSTIYADKDQIRAANDRSSSKQLKQSSAQISSGPEGTSVSSKDRQYSNDKRSKSDAYVGRDGTVAYSNKDSEKTSRQSNTNYADQNSVRSDSAASDQTSKSYDRGYSDKNIVAHSSGSRGSQNQKSSSYRADKDGFSSSTNTEKSKFSSSNSVVETSDGASASRESSAEDTKSSNSNVQSDEKSASQSSSSRSSQESASYSSSSSSSTLSEDSSEVDIDLGNLGWWWNSDNKVQRAAGGATKSGASSSTQATTVSGADDSADSYTWWWNPRRSSSSSSSASSSSSGSNVGGSSQSSGSSTSGSNARGHLGTVSSTGSTSNTDSSSKSAGSRTSGGSSTYGYSSSHRGGSVSSTGSSSNTDSSTKNAGSSTSGGSSTYGYSSSHRGGSVSSTGSSSNTDSSTKSAGSSTSGGSSTYGYSSRHRGGRVSSTGSSSTTDASSNSVGSSTSGGSSTYGYSSNSRDGSVSSTGSSSNTDSNSNSAGSSTSGGSSTYGYSSNSRDGSVSSTGSSSNTDSNSNSAGSSTSGGSSTYGYSSNSRDGSVSSTGSSSNTDASTDLTGSSTSGGSSTYGYSSDSRDGSVSSTGSSSNTDASTDLAGSSTSGGSSTYGYSSDCGDGSVSSTGSSSNTDASTDLAGSSTSGGSSTYGYSSDSRDGSVSSTGSSSNTDASTDLAGSSTSGGSSTYGYSSNSRDGSVSSTGSSSNTDASTDLTGSSTSGGSSTYGYSSSNRDGSVLATGSSSNTDASTTEESTTSAGSSTEGYSSSSHDGSVTSTDGSSTSGGASSSSASTAKSDAASSEDGFWWWNRRKSGSGHKSATVQSSTTDKTSTDSASSTDSTSSTSGASTTTSGSSSTSGGSSTSDASSTSSSVSRSHHSGVNRLLHKPGQGKICLCFENIFDIPYHLRKNIGV

SEQ ID NO: 10 - protein, 900 aa, Bombyx mori
MKIPYVLLFLVGVAVVNALPNPLFGGLVKSLSKKKQIFEDKFENLKENVGEKFENLKENVGEKVENLKENVGEKLENIKEKAGEKFENLKDNVGEKFENLKDNVGDKLEAAKEKAGEIKKKLVDVGEDLKDELTEDKKIKISISKDEGLTLEKEGYKSDYDRNEYEERGSEHQEDNDSDGSYSKGSEYEKYGEEEKYEERRTHDKFSIGKNGISAERTKSKRGERKEVEGEYEKDYERKENNGGSSEYSERERESLEKSKERYGEQSSKSFSLGKSGLKKQDNSKSYSDKEESKLEKEKKYEKKTKINNERQLDEDENERRTVVGRDEQRQDDQSRDDQSRDDQSQDEETGSDDSDKNRGKDTDDKYSETGTNKSSETKTGKRDGSKSGVTVEREKSESNKKSREFENKEAESSTYRDKNRSVNSGSERKSSGKDEEYSEQNSSNKSFNDGDASADYQTKSKKVEKNSARDKKEKEKTDTRNSDGTYKTSEREKEQSSRVNQSKGSNSRDSSESDKSGRKVNKETETYSDKDAQTSESERTQSKDNGGLKEKSSQSKNYGRDEKYSEEKERSSTGKFGSNDSRARSTKAEEEHVRKSQEETHSEQREKTRSDGVTKYNDGDEHFDSDDTEKTKPNGRSPSHKDTEKAKPNDRSSSDKDTEKTFDKNIDNKRPKDGSSSDKNVEQERENYKSESSRNEFENQKSAHSRYEDNGGLKEKSSQSKNYGRDEKYSEEKERSSTGKSGSNDSRARSTKAEEEHVRKSQEETHSEQRGRTRSDGATTSNDNDKQYDSDDKNNSSTKHKKTVMRSEQSDSSQNENSTSESKKFAKTDGSNKYEAESSSHKQQEARKQSNRVVEKSTDGDNEESYRSESSSSSSSSSSSSRSSSSSTYTGSHDDSSEE

SEQ ID NO: 11 - protein, 1271 aa, Bombyx mori
MNCKVALFLIVAIVAVQALPCQESRPRRCGYRYGCGGGRGRGSGVRRLDSGADDNQGRSCLVGGSAESNDEDSDDRLSVINNTEGHATSSSFDEQSSSARQSSSSYQSQSYNKDSSSTNESSDGGSGSGRTGSAGENGENSDDSSGATKGNSSKSSSSSQGQSASSSSSDEKSSQSSSNSSNNSKSSSQSSSSQNSSGSKGSGSEESSNGGSGSGRTGSAGGTDEDSDDSSGATKGNSSKSSSSSQGQSASSSSSDENSSQSSSNSSNNSKSSSQSSSGQNSSGSKGSGSEESSNGGSGSGRNGSVGGTDEDSDDSSGATKGNSSKSSSSSQGQSASSSSSDEKSSQSSSNSSNNSKSSSQSSSGQNSSGSKGSGSEESSNGGSGSGRNGSAGGTDEDSDDSSGATKGNSSKSSSSSQGQSASSSSSDEKSSQSSSNSSNNSKSSSQSSSGQNSSGSKGSGSEESSNGGSGSGRNGSAGGTDEDSDDSSGATKGNSSKSSSSSQGQSASSSSSDEKSSQSSSNSSNNSKSSSQSSSGQNSSGSKGSGSEESSNGGSGSGRTGSAGETDEDSDDSSGATKGNSSKSSSSSQGKSASSSSSDEKSSQSSSNSSNNSKSSSQSSSSNNSSGSKGSGSEESSNGGSGSGRTGSAGGSDEDSDDSSGATKGNSSKSSSSSQGQSASSSSSDEKSSQSNSNSSNNSKSSSQSSSSNNSSGSKGSGSEESSNGGSGSGRTGSAGGTDEDSDDSSGATKGNSSKSSSSSQGKSASSSSSDEKSSQSSSNSSNNSKSSSQSSSSKNSSGSKGSGSEESSNGGSGSGRTGSAGGTDEDSDDSSGATKGNSSKSSSSSQGKSASSSSSDEKSSQSNSNSSNNSKSSSQSSSSKNSSGSKGSGSEESSNGGSGSGRTGSAGGTDEDSDDSSGATKGNSSKSSSSSQGKSASSSSSNEKSSQSSSNSSNNSKSSSQSSSSKNSSGSKGSGSSESGDKKSSSRGSSGDNSDDDQTDSARSNSKRSTSSDASTKKSSSRKSSNHRSSRSQQAHSSSSKQAQSSSSQQAQNSRSQQAHSSRSQQAHSSSSKQAQSSSSKQAQSSSSKQAQSSSSKQAQSSSSQQAQSSRSQQAHSSRSQQAHSSSSKQAQSSSSKQAQSSSSKQAQSSSSKQAQSSSSQQAQSSRSQQAHSSRSETDSKSSNSGGHSNHSSRTEQKSSAKAISSSEQSQNFSSSSQKSAEAADGSESTQSASEYSSSQSKASSSFSASSASESSSLSSDVNFEEKTD

SEQ ID NO: 12 - protein, 748 aa, Nephila clavipes
QGAGAAAAAAGGAGQGGYGGLGGQGAGQGGYGGLGGQGAGQGAGAAAAAAAGGAGQGGYGGLGSQGAGRGGQGAGAAAAAAGGAGQGGYGGLGSQGAGRGGLGGQGAGAAAAAAAGGAGQGGYGGLGNQGAGRGGQGAAAAAAGGAGQGGYGGLGSQGAGRGGLGGQGAGAAAAAAGGAGQGGYGGLGGQGAGQGGYGGLGSQGAGRGGLGGQGAGAAAAAAAGGAGQGGLGGQGAGQGAGASAAAAGGAGQGGYGGLGSQGAGRGGEGAGAAAAAAGGAGQGGYGGLGGQGAGQGGYGGLGSQGAGRGGLGGQGAGAAAAGGAGQGGLGGQGAGQGAGAAAAAAGGAGQGGYGGLGSQGAGRGGLGGQGAGAVAAAAAGGAGQGGYGGLGSQGAGRGGQGAGAAAAAAGGAGQRGYGGLGNQGAGRGGLGGQGAGAAAAAAAGGAGQGGYGGLGNQGAGRGGQGAAAAAGGAGQGGYGGLGSQGAGRGGQGAGAAAAAAVGAGQEGIRGQGAGQGGYGGLGSQGSGRGGLGGQGAGAAAAAAGGAGQGGLGGQGAGQGAGAAAAAAGGVRQGGYGGLGSQGAGRGGQGAGAAAAAAGGAGQGGYGGLGGQGVGRGGLGGQGAGAAAAGGAGQGGYGGVGSGASAASAAASRLSSPQASSRVSSAVSNLVASGPTNSAALSSTISNVVSQIGASNPGLSGCDVLIQALLEVVSALIQILGSSSIGQVNYGSAGQATQIVGQSVYQALG

SEQ ID NO: 13 - protein, 627 aa, Nephila clavipes
PGGYGPGQQGPGGYGPGQQGPSGPGSAAAAAAAAAAGPGGYGPGQQGPGGYGPGQQGPGRYGPGQQGPSGPGSAAAAAAGSGQQGPGGYGPRQQGPGGYGQGQQGPSGPGSAAAASAAASAESGQQGPGGYGPGQQGPGGYGPGQQGPGGYGPGQQGPSGPGSAAAAAAAASGPGQQGPGGYGPGQQGPGGYGPGQQGPSGPGSAAAAAAAASGPGQQGPGGYGPGQQGPGGYGPGQQGLSGPGSAAAAAAAGPGQQGPGGYGPGQQGPSGPGSAAAAAAAAAGPGGYGPGQQGPGGYGPGQQGPSGAGSAAAAAAAGPGQQGLGGYGPGQQGPGGYGPGQQGPGGYGPGSASAAAAAAGPGQQGPGGYGPGQQGPSGPGSASAAAAAAAAGPGGYGPGQQGPGGYAPGQQGPSGPGSASAAAAAAAAGPGGYGPGQQGPGGYAPGQQGPSGPGSAAAAAAAAAGPGGYGPAQQGPSGPGIAASAASAGPGGYGPAQQGPAGYGPGSAVAASAGAGSAGYGPGSQASAAASRLASPDSGARVASAVSNLVSSGPTSSAALSSVISNAVSQIGASNPGLSGCDVLIQALLEIVSACVTILSSSSIGQVNYGAASQFAQVVGQSVLSAF

(19) United States
(12) Patent Application Publication
(10) Pub. No.: US 2023/0380956 A1
(43) Pub. Date: Nov. 30, 2023

(54) COATED HAIR AND USES THEREOF

(71) Applicant: Georg STRAUCH, Ladenburg (DE)

(72) Inventor: Georg STRAUCH, Ladenburg (DE)

(21) Appl. No.: 18/249,617

(22) PCT Filed: Oct. 19, 2021
(86) PCT No.: PCT/EP2021/078898

§ 371 Date: Apr. 19, 2023

(30) Foreign Application Priority Data
Oct. 20, 2020 (EP) 20202744.7

(51) Int. Cl. A61F 2/10; A61K 8/64; A61Q 5/00; A61L 27/28; A61L 27/36

(52) U.S. Cl. CPC A61F 2/10; A61K 8/64; A61Q 5/00; A61L 27/28; A61F 2220/0008; A61L 27/3666; A61L 2430/18; A61L 2420/02; A61F 2250/0081; A61L 27/3604

(57) ABSTRACT

The present invention relates to implantable hair which is characterized in that it is coated at least in an implantable section with a composition that comes into direct or indirect contact with the hair and contains at least one silk protein. The present invention is further concerned with methods of producing such an implantable hair and the cosmetic use thereof.

The present invention relates to the field of hair transplantation from endogenous hair, exogenous hair or synthetic hair. The present invention describes implantable hair coated at least in an implantable section with a composition that comes into direct contact with the hair and contains at least one silk protein. The present invention is further concerned with methods of producing such implantable hair and the cosmetic use thereof.

Women and men have been confronted with the problem of hair loss for millennia. The hair becomes diminished or falls out. There are numerous reasons for this, which include, in particular, genetic makeup, hormonal changes, stress, environmental effects, symptoms of disease, chemotherapy, hair being torn out, chemical treatment of the hair, colouring of the hair, age-related changes, incorrect or inadequate nutrition, accidents and many other causes.

Accidents such as abrasions, necrosis and burns, often combined with scar formation, also often lead to irksome reduction of hair coverage on parts of the head or body. Specifically asymmetric loss of hair can have a disfiguring effect. Moreover, for emphasis of male body features, greater hair coverage, for instance in the region of the beard or chest, may also be of interest. This may be of particular interest, for instance, in the case of a gender transition from a body with feminine appearance to a body with masculine appearance.

Treatments to counter hair loss, including medical and non-medical methods, such as cosmetic methods, frequently lead to inadequate results, and are not usable by everyone and/or frequently have side effects. Existing treatments to counter hair loss frequently do not meet people's high demands and expectations.

Moreover, they frequently have drawbacks that affect people's physical and mental health. Social factors can also further aggravate the psychological strain that people experience. Hair loss can lead to physiological and emotional problems that people are unable to deal with. These can add up to an emotional trauma and lead even further, resulting in a negative spiral.

A treatment or solution to the hair problem that better meets demands and expectations, especially with regard to greater naturalness, fewer risks and side effects, more comfortable care and/or good cost-benefit ratio, would distinctly improve people's quality of life. Those people would feel healthier or less disadvantaged, they would possibly gain greater social recognition and be reduced to their hair loss to a lesser extent, and they would have less stress and fewer negative feelings. This can have a positive effect on the life of the people affected, including from a social, emotional and financial point of view. The demand for such a hair product for humans and possibly animals appears to be high.

A classical means of visual achievement of complete hair coverage is the use of wigs and toupees. However, a natural appearance and normal, flexible everyday life are often not achievable even with wigs and toupees. Although there have already been advances in their development and quality in the last few years, the suggestions come closer to concealing the hair problem than to a true solution. This is because the edge of the mesh on which the hair is secured is clearly perceptible by one's fingers. This means that both the person wearing wigs and toupees and other people who touch their hair can never forget that the solution is unnatural. Moreover, wigs and toupees have to be taken off time and again and prepared again for use. Therefore, adhesive residues have to be removed, and the hairpiece has to be washed, styled and prepared for adhesive attachment. People who use these means have to correspondingly adjust their everyday life. Since the hair in wigs and toupees has usually been secured to the mesh by knotting, it can break off after a certain time at the knot points subject to high stress. This is aggravated, for instance, by care routines such as hair washing and combing. The edge of the mesh also loses stability with time, since it is subjected to stress on parting of the adhesives and on cleaning. This forms frayed areas where the hair is no longer held.

During the regular changing of wigs and toupees, there are periods of time in which these people are indisposed. In that case, it will not always be possible to attend a spontaneous meeting among friends or work colleagues.

Travel and sporting activities such as swimming and others are also possible only to a limited degree since wigs offer sufficient hold only for a limited time. Furthermore, perspiration cannot easily evaporate, which leads to pruritus. Body heat can also escape only to a limited degree, which can frequently result in headaches.

Moreover, bacteria and other microbes can also colonize wigs and toupees, which can be detrimental to health and cause odours to develop. A possible reaction to adhesive, such as skin irritation or allergy, can also have the effect that wigs are displaced and not secured again. In economic terms, the service life of wigs is additionally limited in spite of care, which also restricts life from a technical and financial point of view. The use of wigs and toupees is thus not a satisfactory solution for many people.

Further means with the aim of reducing or concealing hair loss have been developed. These especially include hormonal treatments by medicaments, transplantations of endogenous hair, implantations of synthetic hair, point tattooing for simulation of short hair, and further methods. In the case of many methods, there is a lack of scientific evidence of efficacy, or there is occurrence of considerable side effects that can adversely affect the wellbeing of the person treated. There is therefore a particular interest in the implantation of hair. These implants are anchored sustainably and in a nature-like manner in the skin (for instance the scalp or the skin of the face).

For instance, the implantation of endogenous hair together with its roots is fundamentally a technical solution that brings many advantages. However, such a method is complex and costly. Moreover, the amount of hair available for implantation is only as much as has been removed beforehand elsewhere from the individual, for instance the back of the head. Therefore, the available resource of endogenous hair is significantly limited. Complete baldness cannot be treated adequately by means of transplantation of endogenous hair. After the transplantation, the hair roots additionally suffer a shock, which at first causes implanted hair to fall out.

Only after a period of months does the hair start to grow there again, with a certain percentage not withstanding the procedure and being lost. The result is thus unpredictable and cannot reliably meet people's expectations. A persistent, unwanted scar after the removal of endogenous hair roots at the back of the head is also possible, depending on the method.

Without a protective layer, however, it is not possible for endogenous hair without roots to be implanted without non-negligible unwanted side effects since it can lead to an immune reaction and inflammation as in the case of ingrowing hair (a common occurrence in the case of curly hair).

Exogenous or synthetic hair, especially without hair roots, would be available as a (virtually) unlimited resource.

The implantation of synthetic hair frequently cannot meet the demand of a natural appearance since the hair consists, for example, of polyester that does not approximate to natural hair to a desired degree in terms of its look and texture. The recipient's body also frequently recognizes exogenous or synthetic hair as a foreign body and reacts accordingly. There can be a considerable, unwanted immune reaction. Owing to the continuing immune reaction in the skin, there can be inflammation and under some circumstances even necrosis. As a result, the nerve ends in the skin can be damaged, which can lead to a loss of feeling at the sites affected.

In order to suppress the immune reaction, implantable synthetic hair is coated with chemical compositions, for example with silver salts. However, this has adverse effects on physical health since the immune system can be actively inhibited, and immune reactions directed against the synthetic hair implants can nevertheless still occur. As soon as the protective, optionally immunosuppressive composition has diffused away from the implanted hair, and hence is no longer present in sufficient concentration, the implanted synthetic or exogenous hair is usually rejected. The anchoring of a rootless and hence smooth synthetic or exogenous hair also frequently leads to inadequate anchoring.

Attempts have therefore been made to mechanically improve securing of hair in the skin by means of different anchorings. For instance, GB 1,504,258 describes hook-shaped anchorings, while GB 2,006,018 teaches loop-shaped anchorings. WO 2011/064772 teaches different forms of anchoring structures, and additionally that multiple individual hairs can also be anchored in the skin via a common anchoring structure. Over and above mechanical anchoring, there are descriptions of structures that address the collagen system, as in U.S. Pat. No. 10,561,490 and WO 2020/180682. However, the anchoring is achieved largely mechanically via surface structures in the implanted section.

In order to reduce any immune reaction, inert coatings of the sections of the hair to be implanted have been described, such as coatings with gold; see U.S. Pat. No. 4,517,997. In the case of an inert coating, however, the hair is held purely mechanically in the skin (for example the scalp) and is not provided with nutrients, nor is it able to grow. Mass transfer with the surrounding dermal tissue is frustrated by the barrier layer of inert material. The removal of worn-out hair has to be achieved by means of comparatively harsh mechanical methods, by tearing the synthetic root, usually made of hard inert material, out of the skin, which can be associated with considerable unwanted stress.

In US-A 2003/195625, hair was coated only comparatively thinly with a barrier layer of inert material, for instance gold, and surrounded with a thickened structure of bioabsorbable material for mechanical anchoring. This achieves the effect that the hair falls out again in a controlled manner after a certain time when the bioabsorbable material has been sufficiently degraded. This obviates the need for harsh mechanical removal of the worn-out hair, but does not achieve permanent anchoring of hair. The adjoining barrier layer of inert material in the implantable section of the hair also prevents direct interaction and mass transfer between the hair and the surrounding dermal tissue. It is not possible for the hair to grow in and take up nutrients from the surrounding dermal tissue. Moreover, the use of two layers, including an inert layer of gold, is costly and technically possible only with considerable extra labour.

There is therefore a need to provide a hair implant that has good compatibility within the dermal tissue and is simultaneously easily producible. The hair implant is to be able to remain within the skin for a prolonged period of time and, after a certain time, to enable mass transfer with the recipient's surrounding dermal tissue.

It has been found, surprisingly, that a coating with a composition containing one or more silk proteins makes it possible to obtain implantable hair having particularly good properties. Such implantable hair is technically easily producible. It can be anchored in the skin with good compatibility. It can also remain in the skin over a prolonged period. Mass transfer with the surrounding dermal tissue is enabled.

Overall, spider silk in particular is notable for its antibacterial and antifungicidal action. It promotes wound healing, and is water-resistant, very heat-stable and biodegradable. The present invention relates to implantable hair which is characterized in that it is coated in at least an implantable section with a composition containing at least one silk protein.

One aspect of the present invention relates to implantable hair coated at least in an implantable section with a composition that comes into direct contact with the hair and contains at least one silk protein.

As used herein, the implantable section is the section of the hair that can be implanted. It is typically implanted into the skin. In one embodiment, it is implanted into the scalp. In another embodiment, it is implanted into one or more other regions of the face or of the human or animal body. For instance, implantation can be effected in the region of one or both eyebrows, in the region of one or both eyelashes, in the moustache region, in the chin region of the beard, in the cheek region of the beard, in the region of one or both sideburns, in the chest region, or in the region of one or both legs. The present invention is suitable for creating new hair coverage or re-establishing hair coverage (hair restoration).

As used herein, the expression “in direct contact with the hair” should be understood in its broadest sense. For instance, the coating is preferably not merely one that merely coats a macroscopic substance that in turn is in contact with the hair. It will be understood that the hair may optionally also itself be coated and/or coloured and/or differently altered. Therefore, direct contact in the context of the present invention may also be with coated hair. It will thus be appreciated that a thin layer (preferably not thicker than 0.5 mm, more preferably not thicker than 0.1 mm, especially not thicker than 0.05 mm) of another material may optionally occur between the hair and the composition. In this case, the person skilled in the art will also understand this as meaning coming into direct contact with the hair (coated in that case). Such an intermediate coating can optionally improve the adhesion of the composition and/or of the silk protein to the hair. If there is such an optional intermediate coating, it is preferably bioabsorbable (biodegradable). If there is such an optional intermediate coating, it is preferably not inert. It is preferable that an optionally present intermediate coating does not disrupt or prevent permanent hair anchoring. An optionally present intermediate coating preferably does not disrupt or prevent mass transfer between hair and surrounding dermal tissue.

In particular embodiments, the composition may also come into immediate contact with the hair without the presence of any interlayer.

The expression “in direct contact with the hair” is preferably characterized in that there is no layer between the hair and the composition that is thicker than 0.5 mm, more preferably not thicker than 0.1 mm, especially not thicker than 0.05 mm.

In a preferred embodiment, the expression “in direct contact with the hair” is characterized in that there is no biologically inert layer between the hair and the composition, especially where the layer is not thicker than 0.5 mm, more preferably not thicker than 0.1 mm, especially not thicker than 0.05 mm.

In a particularly preferred embodiment, there is no interlayer. Therefore, the hair (and therefore its keratin structure) is more preferably in direct contact with the composition as defined herein.

The coating with a composition containing at least one silk protein may also be regarded as a protective layer. This achieves particularly stable implantation into the skin. Such a coating, especially in the case of implantation of the implantable hair under (largely) sterile conditions, also enables protection from bacteria and other microbes that can occur on or in skin and/or hair, and the reduction of unwanted immune reactions, for example inflammation.

In order to utilize the function of the promotion of wound healing by the spider silk, the anchoring containing at least one silk protein may additionally have a readily removable and/or bioabsorbable outer layer containing at least one silk protein. After implantation, the body rapidly breaks down the readily removable and/or bioabsorbable layer and transports it to the epidermis, hence ensuring faster and low-infection wound healing. In this context, “readily removable and/or bioabsorbable” should be understood in its broadest sense such that this layer can be removed mechanically and/or biologically (also: degraded), especially in the body of an individual. It can be bioabsorbed in the process. This may optionally be an uptake into tissue and/or body cells and/or extracellular metabolism. It is also optionally possible for degradation products to be metabolized further in the body and/or secreted from the body.

Silk protein is regularly not perceived as a foreign body. It is possible here for the protective layer made of the composition to at least partly divide the implantable hair from the surrounding tissue in order to reduce or prevent an immune reaction. It is also possible to reduce or prevent bacterial infections and inflammation at the exit site of the implanted hair by means of the protective layer made of the composition. It is possible to prevent bacteria and other microbes from penetrating into the site where the implantable hair is implanted, or from colonizing the implantable hair.

By contrast with wigs, the sebum can additionally be transferred from the skin (for instance the scalp) by a natural route to the hair (naturally occurring and/or implanted hair), without getting caught in the mesh of wigs or toupees. This can protect the hair (naturally occurring and/or implanted hair) from environmental influences, physical effects and/or drying out.

The hair structure can be stabilized. For additional protection, it is possible to coat implantable hair with the composition containing at least one silk protein not just at the implanted site but also completely. This can additionally simplify and improve the styling of the hair.

The coating of the implantable section of the implantable hair with the composition containing at least one silk protein may comprise the entire implantable section or a portion thereof. The coating of the implantable section of the implantable hair with the composition containing at least one silk protein preferably encompasses at least 25%, at least 50%, at least 75%, at least 90% or at least 95% of the surface area of the implantable section. More preferably, the coating of the implantable section of the implantable hair with the composition containing at least one silk protein encompasses the entire implantable section.

The coating of the implantable section of the implantable hair with the composition containing at least one silk protein may optionally also include the exit site of the implantable hair from the recipient's skin after implantation. In this way, it is possible to protect the exit site from bacteria and other microbes and/or to reduce or prevent an immune reaction here too. In one embodiment of the present invention, the coating comprises the implantable section (for instance a real or synthetic hair root and/or a real or synthetic hair follicle and/or a real or synthetic hair bulb) and the exit site of the implantable hair from the skin after implantation.

In one embodiment of the present invention, the composition comprises one or more silk proteins to an extent of at least 10% by weight, to an extent of at least 25% by weight, to an extent of at least 50% by weight, to an extent of at least 75% by weight, to an extent of at least 80% by weight, to an extent of at least 90% by weight, to an extent of at least 95% by weight or to an extent of at least 99% by weight, based on the dry mass of the composition.

In one embodiment of the present invention, the composition comprises one or more silk proteins to an extent of at least 10% by weight, to an extent of at least 25% by weight, to an extent of at least 50% by weight, to an extent of at least 75% by weight, to an extent of at least 80% by weight, to an extent of at least 90% by weight, to an extent of at least 95% by weight or to an extent of at least 99% by weight, based on the total mass of the composition.

The composition used as coating and containing at least one silk protein may optionally also consist of one or more silk proteins and optionally the (cosmetically compatible) salts thereof.

As used herein, “cosmetically compatible” should be understood to mean acceptable in the implantation of the implantable hair in the skin.

The composition used as coating may be solid, pasty or liquid. It may optionally contain a cosmetically compatible solvent in addition to the at least one silk protein. Such a cosmetically compatible solvent may be selected, for example, from the group consisting of water, a cosmetically compatible liquid buffer, a hydroalcoholic mixture containing water and up to 5% by volume of ethanol, dimethyl sulfoxide (DMSO) and combinations of two or more of these.

Optionally, the composition used as coating may also contain one or more preservatives, one or more antibacterial (for instance bactericidal and/or bacteriostatic) agents, one or more antifungicidal agents, one or more antiviral agents, haemostatic agents, one or more vitamins, one or more bioactive substances that promote hair growth or perfusion, one or more local anaesthetics, one or more thickeners, one or more hormones, one or more acid regulators, or a combination of two or more of the components mentioned.

It is optionally possible for the composition used as coating, irrespective of its ingredients, also to have dried onto the surface of the implantable hair.

Optionally, the composition used as coating may also have partly diffused into the implantable hair. However, preferably more than 50% by weight of the at least one silk protein, based on the total amount of the composition remaining on the hair, remains on the surface of the implantable hair, and less than 50% by weight of the at least one silk protein, based on the total amount in the composition remaining on the hair, diffuses into the hair.

According to the invention, the hair is at least partly in direct contact with the coating. The coating therefore at least partly directly adjoins the hair structure enclosed by this layer. It is thus possible for mass transfer to take place.

The invention generally also relates to hair implants with silk (for instance spider silk and/or caterpillar silk) with medical and aesthetic functionality.

The silk protein may be any known silk protein. In one embodiment, the at least one silk protein used has at least 90% sequence homology or at least 95% sequence homology or at least 98% sequence homology to or sequence identity with an insect silk protein or a spider silk protein.

In one embodiment, the at least one silk protein has at least 90% sequence homology or at least 95% sequence homology or at least 98% sequence homology to or sequence identity with a silk protein from the family of the Bombycidae, the Araneae, for instance of the Mygalomorphae, the Araneomorphae and/or the Mesothelae.

In one embodiment, the at least one silk protein has at least 90% sequence homology or at least 95% sequence homology or at least 98% sequence homology to or sequence identity with a silk protein from one of the genera Bombyx (e.g. Bombyx mori, Bombyx mandarina), Araneus, Nephila, Antheraea (e.g. Antheraea pernyi, Antheraea yamamai, Antheraea mylitta, Antheraea roylei, Antheraea proyeli, Antheraea paphia, Antheraea frithi, Antheraea assama), Arachnura, Caerostris, Argiope, Cyrtophora, Celaenia, Gasteracantha, Ordgarius, Neoscona, Zygiella, Parawixia, Neoscona, Dolophones, Aculeperia, Eriophora, Anepsion, Tegenaria, Heurodes, Cyclosa, Astracantha, Eriovixia, Nephilengys, Herennia, Acusilas, Neoscona, Poltys, Arkys, Poecilopachys, Hyalophora (e.g. Hyalophora cecropia), Samia (e.g. Samia cynthia), Attacus (Attacus atlas), Circula (e.g. Circula trifenestrata), Gonometa (e.g. Gonometa postica, Gonometa rufobrunnea) and/or Anaphe (e.g. Anaphe panda, Anaphe moloneyi).

In one embodiment, the at least one silk protein has at least 90% sequence homology or at least 95% sequence homology or at least 98% sequence homology to or sequence identity with a silk protein from one of the genera Bombyx, Araneus, Nephila, Antheraea, Arachnura, Caerostris, Argiope, Cyrtophora, Celaenia, Gasteracantha, Ordgarius, Neoscona, Zygiella, Parawixia, Neoscona, Dolophones, Aculeperia, Eriophora, Anepsion, Tegenaria, Heurodes, Cyclosa, Astracantha, Eriovixia, Nephilengys, Herennia, Acusilas, Neoscona, Poltys, Arkys, Poecilopachys.

In one embodiment, the silk protein has at least 90% sequence homology or at least 95% sequence homology or at least 98% sequence homology to or sequence identity with a silk protein from a genus selected from the group consisting of Bombyx, Araneus Antheraea and Nephila.

In one embodiment, the at least one silk protein has at least 90% sequence homology or at least 95% sequence homology or at least 98% sequence homology to or sequence identity with a silk protein from a species selected from the group consisting of Bombyx mori, Bombyx mandarina, Antheraea pemyi, Antheraea yamamai, Antheraea mylitta, Antheraea roylei, Antheraea proyeli, Antheraea paphia, Antheraea frithi, Antheraea assama Araneus didenatus, Nephila clavipes, Araneus bicentenarius, Arachnura higginsi, Araneus circulissparsus, Araneus diadematus, Caerostris darwini, Argiope picta, Argiope trifasciata, Nephila antipodiana, Cyrtophora beccarii, Celaenia excavata, Gasteracantha kuhlii, Argiope aurantia, Ordgarius furcatus, Ordgarius magnificus, Neoscona nautica, Neoscona rufofemorata, Zygiella calyptrata, Parawixia dehaani, Neoscona oxancensis, Gasteracantha cancriformis, Gasteracantha arcuata, Cyrtophora moluccensis, Cyrtophora parnasia, Dolophones conifera, Dolophones turrigera, Gasteracantha doriae, Gasteracantha mammosa, Cyrtophora exanthematica, Aculeperia ceropegia, Eriophora pustulosa, Anepsion depressium, Gasteracantha quadrispinosa, Eriophora transmarina, Araneus bicentenarius, Nephila maculata, Gasteracantha hasseltii, Tegenaria atrica, Heurodes turrita, Cyclosa insulana, Astracantha minax, Araneus mitificus, Eriovixia laglaisei, Cyclosa bifida, Nephilengys malabarensis, Argiope versicolor, Herennia ornatissima, Argiope aemula, Cyrtophora unicolor, Cyrtophora hirta, Argiope keyserlingi, Acusilas coccineus, Argiope argentata, Gasteracantha cancriformis, Neoscona domiciliorum, Argiope aetheria, Argiope Keyserlingi, Poltys illepidus, Arkys clavatus, Arkys lancearius, Poecilopachys australasia, Nephila clavipes, Nephila senegalensis, Nephila madagascariensis, Hyalophora cecropia, Samia Samia cynthia, Attacus atlas, Circula trifenestrata, Gonometa postica, Gonometa rufobrunnea, Anaphe panda and/or Anaphe moloneyi.

In one embodiment, the silk protein has at least 90% sequence homology or at least 95% sequence homology or at least 98% sequence homology to or sequence identity with a silk protein from a species selected from the group consisting of Bombyx mori, Antheraea pemyi, Araneus diadematus, Nephila clavipes, Araneus bicentenarius and Caerostris darwini.

In one embodiment, the at least one silk protein has at least 90% sequence homology or at least 95% sequence homology or at least 98% sequence homology to or sequence identity with a silk protein from Bombyx mori.

In one embodiment, the at least one silk protein has at least 90% sequence homology or at least 95% sequence homology or at least 98% sequence homology to or sequence identity with a silk protein from Antheraea pernyi.

In one embodiment, the at least one silk protein has at least 90% sequence homology or at least 95% sequence homology or at least 98% sequence homology to or sequence identity with a silk protein from Araneus diadematus.

In one embodiment, the at least one silk protein has at least 90% sequence homology or at least 95% sequence homology or at least 98% sequence homology to or sequence identity with a silk protein from Nephila clavipes.

In one embodiment, the at least one silk protein has at least 90% sequence homology or at least 95% sequence homology or at least 98% sequence homology to or sequence identity with a silk protein from Araneus bicentenarius.

In one embodiment, the at least one silk protein has at least 90% sequence homology or at least 95% sequence homology or at least 98% sequence homology to or sequence identity with a silk protein from Caerostris darwini.

Further silk proteins usable in accordance with the present invention and their biochemical properties are described in WO 2007/025719, WO 2006/008163, WO 2004/024176, DE-A 10333253, US 2007/196429, US 2007/214520, US 2009/123967 and WO 2011/069643.

In one embodiment of the present invention, the at least one silk protein is characterized in that it is selected from the group consisting of fibroin, sericin, spidroin 1 and spidroin 2. Spidroin 2 may optionally also be “Araneus diadematus Fibroin 3” (ADF-3) or “Araneus diadematus Fibroin 4” (ADF-4), as described, for instance, in WO 2007/025719 and WO 2006/008163. ADF-3 may have a sequence having at least 90% homology or at least 95% homology or at least 98% homology or at least 99% homology to SEQ ID NO: 1 from WO 2006/008163. ADF-4 may have a sequence having at least 90% homology or at least 95% homology or at least 98% homology or at least 99% homology to SEQ ID NO: 2 from WO 2006/008163. ADF-3 may have a sequence as in SEQ ID NO: 1 from WO 2006/008163. ADF-4 may have a sequence as in SEQ ID NO: 2 from WO 2006/008163.

In one embodiment of the present invention, the at least one silk protein comprises at least two, at least three, at least four or more than four sequence sections each having one of the following sequences SEQ ID NO: 1 to 6:

(SEQ ID NO: 1)
GPGXX,
(SEQ ID NO: 2)
GVPGX,
(SEQ ID NO: 3)
GSGXX,
(SEQ ID NO: 4)
GGYXX,
(SEQ ID NO: 5)
PQQXX,
(SEQ ID NO: 6)
GYGXX,

where X may be any natural amino acid, and is preferably selected from the group consisting of V, G, A, S, Y, P and Q.

In one embodiment of the present invention, the at least one silk protein comprises at least two, at least three, at least four or more than four identical sequence sections of a sequence SEQ ID NO: 1 to 6.

Fibroin is a commonly known fibrous protein which is secreted by way of example by the spinning glands of the silk moth (Bombyx mori), but other species too. By way of example, fibroin may comprise a heavy chain of the following sequence SEQ ID NO: 7:

MRVKTFVILCCALQYVAYTNANINDFDEDYFGSDVTVQSSNTTDEIIRDASGAVIEEQITTK
KMQRKNKNHGILGKNEKMIKTFVITTDSDGNESIVEEDVLMKTLSDGTVAQSYVAADAGA
YSQSGPYVSNSGYSTHQGYTSDFSTSAAVGAGAGAGAAAGSGAGAGAGYGAASGAGA
GAGAGAGAGYGTGAGAGAGAGYGAGAGAGAGAGYGAGAGAGAGAGYGAGAGAGAG
AGYGAGAGAGAGAGYGAGAGAGAGAGYGAASGAGAGAGYGQGVGSGAASGAGAGA
GAGSAAGSGAGAGAGTGAGAGYGAGAGAGAGAGYGAASGTGAGYGAGAGAGYGGA
SGAGAGAGAGAGAGAGAGYGTGAGYGAGAGAGAGAGAGAGYGAGAGAGYGAGYGV
GAGAGYGAGYGAGAGSGAASGAGSGAGAGSGAGAGSGAGAGSGAGAGSGAGAGSG
AGAGSGAGAGSGAGAGSGTGAGSGAGAGYGAGAGAGYGAGAGSGAASGAGAGSGA
GAGSGAGAGSGAGAGSGAGAGSGAGAGYGAGAGAGYGAGAGAGYGAGAGVGYGAG
AGSGAASGAGAGSGAGAGSGAGAGSGAGAGSGAGAGSGAGAGSGAGAGSGAGAGS
GAGAGSGAGVGYGAGVGAGYGAGYGAGAGAGYGAGAGSGAASGAGAGAGAGAGTG
SSGFGPYVANGGYSRSDGYEYAWSSDFGTGSGAGAGSGAGAGSGAGAGSGAGAGSG
AGAGSGAGAGYGAGVGVGYGAGYGAGAGAGYGAGAGSGAASGAGAGSGAGAGSGA
GAGSGAGAGSGAGAGSGAGAGSGAGAGSGAGAGSGAGAGSGAGAGSGAGVGSGAG
AGSGAGAGVGYGAGAGVGYGAGAGSGAASGAGAGSGAGAGSGAGAGSGAGAGSGA
GAGSGAGAGSGAGAGSGAGAGSGAGAGSGAGVGYGAGVGAGYGAGYGAGAGAGYG
AGAGSGAASGAGAGSGAGAGSGAGAGSGAGAGSGAGAGSGAGAGSGAGAGSGAGA
GSGAGAGSGAGAGSGAGAGSGAGAGYGAGAGAGYGAGYGAGAGAGYGAGAGSGAA
SGAGSGAGAGSGAGAGAGSGAGAGSGAGAGSGAGAGSGAGAGSGAGAGSGAGAGY
GAGVGAGYGAGYGAGAGAGYGAGAGSGAASGAGAGSGAGAGSGAGAGSGAGAGSG
AGAGSGAGAGSGAGAGSGAGVGYGAGYGAGAGAGYGAGAGSGAASGAGAGAGAGA
GTGSSGFGPYVAHGGYSGYEYAWSSESDFGTGSGAGAGSGAGAGSGAGAGSGAGAG
SGAGYGAGVGAGYGAGYGAGAGAGYGAGAGSGAGSGAGAGSGAGAGSGAGAGSGA
GAGSGAGAGSGAGAGSGAGAGSGAGAGSGAGAGYGAGYGAGAGAGYGAGAGSGAG
SGAGAGSGAGAGSGAGAGSGAGAGSGAGAGSGAGAGSGAGAGSGAGAGYGAGVGA
GYGAGYGAGAGAGYGAGAGSGAGSGAGAGSGAGAGSGAGAGSGAGVGSGAGAGSG
AGAGSGAGAGSGAGAGYGAGYGAGAGAGYGAGAGSGAGSGAGAGSGAGAGSGAGA
GSGAGAGSGAGAGSGAGAGSGAGAGSGAGVGYGAGVGAGYGAGYGAGAGAGYGAG
AGSGAASGAGAGAGAGAGTGSSGFGPYVANGGYSGYEYAWSSESDFGTGSGAGAGS
GAGAGSGAGAGSGAGAGSGAGAGYGAGYGAGAGAGYGAGAGSGAGSGAGAGSGAG
AGSGAGAGSGAGAGSGAGAGSGAGAGSGAGAGSGAGSGSGAGAGSGAGAGSGAGA
GYGAGVGAGYGVGYGAGAGAGYGAGAGSGAASGAGAGAGAGAGTGSSGFGPYVAH
GGYSGYEYAWSSESDFGTGSGAGAGSGAGAGSGAGAGSGAGAGSGAGAGSGAGAG
SGAGAGYGAGVGAGYGAAYGAGAGAGYGAGAGSGAASGAGAGSGAGAGSGAGAGS
GAGAGSGAGAGSGAGAGSGAGAGSGAGAGSGAGAGSGAGAGSGAGAGYGAGAGAG
YGAGAGSGAGSGAGAGSGAGAGSGAGAGSGAGAGSGAGAGSGAGSGSGAGAGSGA
GAGSGAGAGYGAGVGAGYGAGYGAGAGAGYGAGAGSGAGSGAGAGSGAGAGYGAG
AGAGYGAGYGAGAGAGYGAGAGTGAGSGAGAGSGAGAGSGAGAGSGAGAGSGAGA
GSGAGAGSGAGSGSGAGAGSGAGAGSGAGAGSGAGAGSGAGAGSGAGAGYGAGAG
AGYGAGYGAGAGAGYGAGAGSGAGSGAGAGSGAGAGSGAGAGSGAGAGYGAGYGA
GAGSGAASGAGAGAGAGAGTGSSGFGPYVAHGGYSGYEYAWSSESDFGTGSGAGAG
SGAGAGAGAGAGSGAGAGYGAGVGAGYGAGYGAGAGAGYGAGAGSGTGSGAGAGS
GAGAGYGAGVGAGYGAGAGSGAAFGAGAGAGAGSGAGAGSGAGAGSGAGAGSGAG
AGSGAGAGYGAGYGAGVGAGYGAGAGSGAASGAGAGSGAGAGSGAGAGSGAGAGS
GAGAGSGAGAGYGAGVGAGYGAGYGAGAGAGYGAGAGSGAASGAGAGSGAGAGAG
SGAGAGSGAGAGSGAGAGSGAGSGAGAGSGAGAGSGAGAGYGAGAGSGAASGAGA
GAGAGAGTGSSGFGPYVANGGYSGYEYAWSSESDFGTGSGAGAGSGAGAGSGAGAG
SGAGAGSGAGAGYGAGVGAGYGAGYGAGAGAGYGAGAGSGAGSGAGAGSGAGAGS
GAGAGSGAGAGSGAGAGSGAGAGSGAGAGYGAGAGSGAASGAGAGSGAGAGSGAG
AGSGAGAGSGAGAGSGAGAGSGAGAGYGAGVGAGYGVGYGAGAGAGYGAGAGSGA
GSGAGAGSGAGAGSGAGAGSGAGAGSGAGSGAGAGSGAGAGSGAGAGSGAGSGAG
AGSGAGAGYGVGYGAGAGAGYGAGAGSGAGSGAGAGSGAGAGSGAGAGSGAGSGA
GAGSGAGAGSGAGAGSGAGAGYGAGVGAGYGVGYGAGAGAGYGAGAGSGAGSGAG
AGSGAGAGSGAGAGSGAGAGSGAGAGSGAGAGSGAGAGSGAGSGAGAGSGAGAGS
GAGAGSGAGAGSGAGSGAGAGSGAGAGSGAGAGSGAGAGYGAGVGAGYGVGYGAG
VGAGYGAGAGSGAASGAGAGSGAGAGAGSGAGAGSGAGAGSGAGAGSGAGAGSGA
GAGSGAGAGYGAGYGAGVGAGYGAGAGVGYGAGAGAGYGAGAGSGAASGAGAGAG
SGAGAGTGAGAGSGAGAGYGAGAGSGAASGAGAGAGAGAGTGSSGFGPYVANGGYS
GYEYAWSSESDFGTGSGAGAGSGAGAGSGAGAGSGAGAGSGAGAGYGAGVGAGYG
AGAGSGAGSGAGAGSGAGAGSGAGAGSGAGAGSGAGAGYGAGAGSGTGSGAGAGS
GAGAGSGAGAGSGAGAGSGAGAGSGAGAGSGVGAGYGVGYGAGAGAGYGVGYGAG
AGAGYGAGAGSGTGSGAGAGSGAGAGSGAGAGSGAGAGSGAGAGSGAGAGSGAGA
GYGAGVGAGYGVGYGAGAGAGYGAGAGSGAGSGAGAGSGAGAGSGAGAGSGAGAG
SGAGSGAGAGSGAGAGSGAGAGSGAGSGAGAGSGAGAGYGVGYGAGAGAGYGAGA
GSGAGSGAGAGSGAGAGSGAGAGSGAGSGAGAGSGAGAGSGAGAGSGAGAGYGAG
VGAGYGVGYGAGAGAGYGAGAGSGAGSGAGAGSGAGAGSGAGAGSGAGAGSGAGA
GSGAGAGSGAGAGSGAGSGAGAGSGAGAGSGAGAGSGAGAGYGAGVGAGYGVGYG
AGAGAGYGAGAGSGAASGAGAGAGAGAGTGSSGFGPYVANGGYSGYEYAWSSESDF
GTGSGAGAGSGAGAGSGAGAGYGAGYGAGVGAGYGAGAGVGYGAGAGAGYGAGAG
SGAASGAGAGAGAGAGSGAGAGSGAGAGAGSGAGAGYGAGYGIGVGAGYGAGAGV
GYGAGAGAGYGAGAGSGAASGAGAGSGAGAGSGAGAGSGAGAGSGAGAGSGAGAG
SGAGAGYGAGYGAGVGAGYGAGAGVGYGAGAGAGYGAGAGSGAASGAGAGAGAGA
GAGSGAGAGSGAGAGSGAGAGSGAGAGSGAGAGSGAGAGSGAGAGSGAGAGSGAG
AGYGAGVGAGYGAGYGGAGAGYGAGAGSGAASGAGAGSGAGAGSGAGAGSGAGAG
SGAGAGSGAGAGYGAGAGSGAASGAGAGAGAGAGTGSSGFGPYVNGGYSGYEYAW
SSESDFGTGSGAGAGSGAGAGSGAGAGYGAGVGAGYGAGYGAGAGAGYGAGAGSG
AASGAGAGSGAGAGSGAGAGSGAGAGSGAGSGAGAGSGAGAGSGAGAGSGAGAGS
GAGAGSGAGAGYGAGVGAGYGAGYGAGAGAGYGAGAGSGAASGAGAGSGAGAGAG
SGAGAGSGAGAGSGAGAGSGAGAGSGAGAGSGAGSGAGAGSGAGAGYGAGYGAGV
GAGYGAGAGVGYGAGAGAGYGAGAGSGAASGAGAGSGSGAGSGAGAGSGAGAGSG
AGAGAGSGAGAGSGAGAGSGAGAGYGAGYGAGAGSGAASGAGAGAGAGAGTGSSG
FGPYVANGGYSGYEYAWSSESDFGTGSGAGAGSGAGAGSGAGAGYGAGVGAGYGAG
YGAGAGAGYGAGAGSGAGSGAGAGSGAGAGSGAGAGSGAGAGSGAGAGSGAGAGS
GAGAGSGAGAGYGAGYGAGAGAGYGAGAGVGYGAGAGAGYGAGAGSGAGSGAGAG
SGSGAGAGSGSGAGSGAGAGSGAGAGSGAGAGSGAGAGSGAGAGSGAGAGSGAGA
GYGAGYGIGVGAGYGAGAGVGYGAGAGAGYGAGAGSGAASGAGAGSGAGAGSGAG
AGSGAGAGSGAGAGSGAGAGSGAGAGSGAGAGSGAGAGSGAGAGYGAGAGVGYGA
GAGSGAASGAGAGSGAGAGSGAGAGSGAGAGSGAGAGSGAGAGSGAGAGSGAGSG
AGAGSGAGAGYGAGYGAGVGAGYGAGAGYGAGYGVGAGAGYGAGAGSGAGSGAGA
GSGAGAGSGAGAGSGAGAGSGAGAGSGAGSGAGAGYGAGAGAGYGAGAGAGYGAG
AGSGAASGAGAGAGAGSGAGAGSGAGAGSGAGSGAGAGSGAGAGYGAGAGSGAAS
GAGAGSGAGAGAGAGAGAGSGAGAGSGAGAGYGAGAGSGAASGAGAGAGAGTGSS
GFGPYVANGGYSRREGYEYAWSSKSDFETGSGAASGAGAGAGSGAGAGSGAGAGSG
AGAGSGAGAGGSVSYGAGRGYGQGAGSAASSVSSASSRSYDYSRRNVRKNCGIPRRQ
LVVKFRALPCVNC

Fibroin may optionally additionally comprise a light chain of the following sequence SEQ ID NO: 8:

MKPIFLVLLVATSAYAAPSVTINQYSDNEIPRDIDDGKASSVISRAWDY
VDDTDKSIAILNVQEILKDMASQGDYASQASAVAQTAGIIAHLSAGIPG
DACAAANVINSYTDGVRSGNFAGFRQSLGPFFGHVGQNLNLINQLVINP
GQLRYSVGPALGCAGGGRIYDFEAAWDAILASSDSSFLNEEYCIVKRLY
NSRNSQSNNIAAYITAHLLPPVAQVFHQSAGSITDLLRGVGNGNDATGL
VANAQRYIAQAASQVHV

Sericin is a well-known protein which, in its natural environment, can hold fibroin strands together. Sericin can be secreted by way of example by the spinning glands of the silk moth (Bombyx mori), but other species too. Sericin may, by way of example, have one of the following sequences SEQ ID NO: 9 to 11:

Sericin 1 may comprise SEQ ID NO: 9:

MRFVLCCTLIALAALSVKAFGHHPGNRDTVEVKNRKYNAASSESSYLNK
DNDSISAGAHRAKSVEQSQDKSKYTSGPEGVSYSGRSQNYKDSKQAYAD
YHSDPNGGSASAGQSRDSSLRERNVHYVSDGEAVAASSDARDENRSAQQ
NAQANWNADGSYGVSADRSGSASSRRRQANYYSDKDITAASKDDSRADS
SRRSNAYYNRDSDGSESAGLSDRSASSSKNDNVFVYRTKDSIGGQAKSS
RSSHSQESDAYYNSSPDGSYNAGTRDSSISNKKKASSTIYADKDQIRAA
NDRSSSKQLKQSSAQISSGPEGTSVSSKDRQYSNDKRSKSDAYVGRDGT
VAYSNKDSEKTSRQSNTNYADQNSVRSDSAASDQTSKSYDRGYSDKNIV
AHSSGSRGSQNQKSSSYRADKDGFSSSTNTEKSKFSSSNSVVETSDGAS
ASRESSAEDTKSSNSNVQSDEKSASQSSSSRSSQESASYSSSSSSSTLS
EDSSEVDIDLGNLGWWWNSDNKVQRAAGGATKSGASSSTQATTVSGADD
SADSYTWWWNPRRSSSSSSSASSSSSGSNVGGSSQSSGSSTSGSNARGH
LGTVSSTGSTSNTDSSSKSAGSRTSGGSSTYGYSSSHRGGSVSSTGSSS
NTDSSTKNAGSSTSGGSSTYGYSSSHRGGSVSSTGSSSNTDSSTKSAGS
STSGGSSTYGYSSRHRGGRVSSTGSSSTTDASSNSVGSSTSGGSSTYGY
SSNSRDGSVSSTGSSSNTDSNSNSAGSSTSGGSSTYGYSSNSRDGSVSS
TGSSSNTDSNSNSAGSSTSGGSSTYGYSSNSRDGSVSSTGSSSNTDAST
DLTGSSTSGGSSTYGYSSDSRDGSVSSTGSSSNTDASTDLAGSSTSGGS
STYGYSSDCGDGSVSSTGSSSNTDASTDLAGSSTSGGSSTYGYSSDSRD
GSVSSTGSSSNTDASTDLAGSSTSGGSSTYGYSSNSRDGSVSSTGSSSN
TDASTDLTGSSTSGGSSTYGYSSSNRDGSVLATGSSSNTDASTTEESTT
SAGSSTEGYSSSSHDGSVTSTDGSSTSGGASSSSASTAKSDAASSEDGF
WWWNRRKSGSGHKSATVQSSTTDKTSTDSASSTDSTSSTSGASTTTSGS
SSTSGGSSTSDASSTSSSVSRSHHSGVNRLLHKPGQGKICLCFENIFDI
PYHLRKNIGV

Sericin 2 may comprise SEQ ID NO: 10:

MKIPYVLLFLVGVAVVNALPNPLFGGLVKSLSKKKQIFEDKFENLKENV
GEKFENLKENVGEKVENLKENVGEKLENIKEKAGEKFENLKDNVGEKFE
NLKDNVGDKLEAAKEKAGEIKKKLVDVGEDLKDELTEDKKIKISISKDE
GLTLEKEGYKSDYDRNEYEERGSEHQEDNDSDGSYSKGSEYEKYGEEEK
YEERRTHDKFSIGKNGISAERTKSKRGERKEVEGEYEKDYERKENNGGS
SEYSERERESLEKSKERYGEQSSKSFSLGKSGLKKQDNSKSYSDKEESK
LEKEKKYEKKTKINNERQLDEDENERRTVVGRDEQRQDDQSRDDQSRDD
QSQDEETGSDDSDKNRGKDTDDKYSETGTNKSSETKTGKRDGSKSGVTV
EREKSESNKKSREFENKEAESSTYRDKNRSVNSGSERKSSGKDEEYSEQ
NSSNKSFNDGDASADYQTKSKKVEKNSARDKKEKEKTDTRNSDGTYKTS
EREKEQSSRVNQSKGSNSRDSSESDKSGRKVNKETETYSDKDAQTSESE
RTQSKDNGGLKEKSSQSKNYGRDEKYSEEKERSSTGKFGSNDSRARSTK
AEEEHVRKSQEETHSEQREKTRSDGVTKYNDGDEHFDSDDTEKTKPNGR
SPSHKDTEKAKPNDRSSSDKDTEKTFDKNIDNKRPKDGSSSDKNVEQER
ENYKSESSRNEFENQKSAHSRYEDNGGLKEKSSQSKNYGRDEKYSEEKE
RSSTGKSGSNDSRARSTKAEEEHVRKSQEETHSEQRGRTRSDGATTSND
NDKQYDSDDKNNSSTKHKKTVMRSEQSDSSQNENSTSESKKFAKTDGSN
KYEAESSSHKQQEARKQSNRVVEKSTDGDNEESYRSESSSSSSSSSSSS
RSSSSSTYTGSHDDSSEE

Sericin 3 may comprise SEQ ID NO: 11:

MNCKVALFLIVAIVAVQALPCQESRPRRCGYRYGCGGGRGRGSGVRRLD
SGADDNQGRSCLVGGSAESNDEDSDDRLSVINNTEGHATSSSFDEQSSS
ARQSSSSYQSQSYNKDSSSTNESSDGGSGSGRTGSAGENGENSDDSSGA
TKGNSSKSSSSSQGQSASSSSSDEKSSQSSSNSSNNSKSSSQSSSSQNS
SGSKGSGSEESSNGGSGSGRTGSAGGTDEDSDDSSGATKGNSSKSSSSS
QGQSASSSSSDENSSQSSSNSSNNSKSSSQSSSGQNSSGSKGSGSEESS
NGGSGSGRNGSVGGTDEDSDDSSGATKGNSSKSSSSSQGQSASSSSSDE
KSSQSSSNSSNNSKSSSQSSSGQNSSGSKGSGSEESSNGGSGSGRNGSA
GGTDEDSDDSSGATKGNSSKSSSSSQGQSASSSSSDEKSSQSSSNSSNN
SKSSSQSSSGQNSSGSKGSGSEESSNGGSGSGRNGSAGGTDEDSDDSSG
ATKGNSSKSSSSSQGQSASSSSSDEKSSQSSSNSSNNSKSSSQSSSGQN
SSGSKGSGSEESSNGGSGSGRTGSAGETDEDSDDSSGATKGNSSKSSSS
SQGKSASSSSSDEKSSQSSSNSSNNSKSSSQSSSSNNSSGSKGSGSEES
SNGGSGSGRTGSAGGSDEDSDDSSGATKGNSSKSSSSSQGQSASSSSSD
EKSSQSNSNSSNNSKSSSQSSSSNNSSGSKGSGSEESSNGGSGSGRTGS
AGGTDEDSDDSSGATKGNSSKSSSSSQGKSASSSSSDEKSSQSSSNSSN
NSKSSSQSSSSKNSSGSKGSGSEESSNGGSGSGRTGSAGGTDEDSDDSS
GATKGNSSKSSSSSQGKSASSSSSDEKSSQSNSNSSNNSKSSSQSSSSK
NSSGSKGSGSEESSNGGSGSGRTGSAGGTDEDSDDSSGATKGNSSKSSS
SSQGKSASSSSSNEKSSQSSSNSSNNSKSSSQSSSSKNSSGSKGSGSSE
SGDKKSSSRGSSGDNSDDDQTDSARSNSKRSTSSDASTKKSSSRKSSNH
RSSRSQQAHSSSSKQAQSSSSQQAQNSRSQQAHSSRSQQAHSSSSKQAQ
SSSSKQAQSSSSKQAQSSSSKQAQSSSSQQAQSSRSQQAHSSRSQQAHS
SSSKQAQSSSSKQAQSSSSKQAQSSSSKQAQSSSSQQAQSSRSQQAHSS
RSETDSKSSNSGGHSNHSSRTEQKSSAKAISSSEQSQNFSSSSQKSAEA
ADGSESTQSASEYSSSQSKASSSFSASSASESSSLSSDVNFEEKTD

Spidroins are fibrous proteins from the silk of some spiders, by way of example Nephila clavipes. Spidroin 1 may comprise, by way of example, the following sequence SEQ ID NO: 12:

QGAGAAAAAAGGAGQGGYGGLGGQGAGQGGYGGLGGQGAGQGAGAAAAA
AAGGAGQGGYGGLGSQGAGRGGQGAGAAAAAAGGAGQGGYGGLGSQGAG
RGGLGGQGAGAAAAAAAGGAGQGGYGGLGNQGAGRGGQGAAAAAAGGAG
QGGYGGLGSQGAGRGGLGGQGAGAAAAAAGGAGQGGYGGLGGQGAGQGG
YGGLGSQGAGRGGLGGQGAGAAAAAAAGGAGQGGLGGQGAGQGAGASAA
AAGGAGQGGYGGLGSQGAGRGGEGAGAAAAAAGGAGQGGYGGLGGQGAG
QGGYGGLGSQGAGRGGLGGQGAGAAAAGGAGQGGLGGQGAGQGAGAAAA
AAGGAGQGGYGGLGSQGAGRGGLGGQGAGAVAAAAAGGAGQGGYGGLGS
QGAGRGGQGAGAAAAAAGGAGQRGYGGLGNQGAGRGGLGGQGAGAAAAA
AAGGAGQGGYGGLGNQGAGRGGQGAAAAAGGAGQGGYGGLGSQGAGRGG
QGAGAAAAAAVGAGQEGIRGQGAGQGGYGGLGSQGSGRGGLGGQGAGAA
AAAAGGAGQGGLGGQGAGQGAGAAAAAAGGVRQGGYGGLGSQGAGRGGQ
GAGAAAAAAGGAGQGGYGGLGGQGVGRGGLGGQGAGAAAAGGAGQGGYG
GVGSGASAASAAASRLSSPQASSRVSSAVSNLVASGPTNSAALSSTISN
VVSQIGASNPGLSGCDVLIQALLEVVSALIQILGSSSIGQVNYGSAGQA
TQIVGQSVYQALG

Spidroin 2 may comprise, by way of example, the following sequence SEQ ID NO: 13:

PGGYGPGQQGPGGYGPGQQGPSGPGSAAAAAAAAAAGPGGYGPGQQGPG
GYGPGQQGPGRYGPGQQGPSGPGSAAAAAAGSGQQGPGGYGPRQQGPGG
YGQGQQGPSGPGSAAAASAAASAESGQQGPGGYGPGQQGPGGYGPGQQG
PGGYGPGQQGPSGPGSAAAAAAAASGPGQQGPGGYGPGQQGPGGYGPGQ
QGPSGPGSAAAAAAAASGPGQQGPGGYGPGQQGPGGYGPGQQGLSGPGS
AAAAAAAGPGQQGPGGYGPGQQGPSGPGSAAAAAAAAAGPGGYGPGQQG
PGGYGPGQQGPSGAGSAAAAAAAGPGQQGLGGYGPGQQGPGGYGPGQQG
PGGYGPGSASAAAAAAGPGQQGPGGYGPGQQGPSGPGSASAAAAAAAAG
PGGYGPGQQGPGGYAPGQQGPSGPGSASAAAAAAAAGPGGYGPGQQGPG
GYAPGQQGPSGPGSAAAAAAAAAGPGGYGPAQQGPSGPGIAASAASAGP
GGYGPAQQGPAGYGPGSAVAASAGAGSAGYGPGSQASAAASRLASPDSG
ARVASAVSNLVSSGPTSSAALSSVISNAVSQIGASNPGLSGCDVLIQAL
LEIVSACVTILSSSSIGQVNYGAASQFAQVVGQSVLSAF

A silk protein and compositions containing such a silk protein or a mixture of silk proteins are optionally commercially available, for instance from AMSilk GmbH (Am Klopferspitz 19 im IZB, 82152 Planegg/Munich, Germany).

A silk protein can be obtained naturally or produced synthetically. A silk protein can be taken from the organism of its original genetic origin, and hence obtained, for instance, from or directly from spiders or caterpillars. It can be taken, for instance, from a silk spider, by way of example as specified above. The obtaining of natural spider silk is technically comparatively difficult and complex. Therefore, a silk protein can alternatively also be expressed heterologously. In order to produce heterologously expressed silk protein, it is possible to take deoxyribonucleic acid (DNA) from the organism of its original genetic origin (for instance a spider or a caterpillar) and transfer it to a host organism (e.g. bacteria, insect cells, mammalian cells, plant cells, fungal cells (e.g. yeast cells)). It is possible here to add promoter sequences and optionally enhancer sequences and/or stop sequences that are recognizable to the host organism. The at least one silk protein can be produced by the cells in cell culture. A silk protein may optionally remain in the host cell, which is then harvested in a batch method or is secreted by the host cell. Such biotechnological methods are well known to the person skilled in the art. Heterologous expression can be effected, for example, in a bacterium, for instance Escherichia coli, as described in US-A 2007/196429, US-A 2007/214520, US-A 2009/123967 and WO 2011/069643.

The at least one implantable section of the hair may lie at any position in the hair. In one embodiment, the implantable section is an implantable terminal end of the hair. For example, the hair may be coated with the composition containing the at least one silk protein and/or be implantable over a length of up to 1 μm, of 1 to 10 μm, of 5 to 50 μm, of 10 to 100 μm, of 50 to 500 μm, of 100 μm to 1 mm, of 0.5 to 5 mm or of more than 5 mm. In that case, the hair can be implanted into the skin, for instance the scalp, by this terminal end.

It is also possible that both terminal ends of the hair are implanted.

In that case, it is optionally possible after implantation to cut open a ring closure formed in that the hair is optionally divided by a cut between the two implanted terminal ends.

In another embodiment, the implantable section is an implantable kink in the middle of the hair or a kinkable section in the middle of the hair.

As understood herein, the “middle” of the hair should be understood in its broadest sense as a section of the hair that is not at a terminal end of the hair, and therefore not at the root or the tip of the hair. The middle of the hair over the length of the hair is preferably in the region of the inner 8/10 of the length of the hair, and therefore in the region not in the outer 10% at either end of the length of the hair. The middle may optionally also be (roughly) in the region of the mathematical middle of the hair (from the root to the tip of the hair) (+/−20%, preferably +/−10%, especially +/−5%).

For instance, the hair may be kinked and may contain a notch in the kink or kinkable section that may be coated with the composition containing the at least one silk protein, for example over a length of up to 1 μm, of 1 to 10 μm, of 5 to 50 μm, of 10 to 100 μm, of 50 to 500 μm, of 100 μm to 1 mm, of 0.5 to 5 mm or of more than 5 mm. This section can then be implanted. In that case, preference is given to implanting a V-shaped kink into the skin by the tip of the V shape.

The implantable hair may be any hair. As used herein, the term “hair” should be understood in its broadest sense to mean a fibre structure having a visual appearance like a human or animal hair, preferably a human hair or that of a mammal, especially a human hair. The implantable hair may optionally have a hair follicle and/or a hair root and/or a hair bulb, or not have any hair follicle or any hair root or any hair bulb.

The implantable hair may be endogenous hair, exogenous hair or synthetic hair. In one embodiment of the present invention, the implantable hair is a human hair. This may be the treated person's own hair or may be exogenous hair from a human or animal donor.

In one embodiment of the present invention, the implantable hair is a hair from the human head. It is also alternatively possible to use other hair from the body, for example chest hair, armpit hair, pubic hair, leg hair, beard hair and/or arm hair.

Optionally, the human implantable hair may have a hair follicle and/or a hair root and/or a hair bulb, or not have any hair follicle or any hair root or any hair bulb. In one embodiment of the present invention, the implantable hair is a cut-off human hair without hair follicle and without hair root and without hair bulb. It is possible here by way of example to use hair from a hairdresser's.

In an alternative embodiment of the present invention, the implantable hair is a synthetic hair or an animal hair. Such hair is described in the prior art. A synthetic hair may also be referred to as artificial hair. Artificial hair may also contain silk or consist of silk.

Implantable hair may have any desired thickness. In one embodiment, the implantable hair has an average diameter of 0.02 to 0.15 mm over the entire length.

It is also possible to use thinner or thicker hair. The hair may have, for example, an average diameter of 0.01 to 0.3 mm, of 0.03 to 0.1 mm, of 0.02 to 0.15 mm, of 0.03 to 0.1 mm, of 0.04 to 0.09 mm, of 0.04 to 0.08 mm, of 0.05 to 0.07 mm or of 0.09 to 0.5 mm.

The coating of the implantable hair with the composition containing at least one silk protein may be of different thickness. It may have a (largely) homogeneous thickness over larger areas or have different thicknesses. In the case of a homogeneous coating, the coating thickness may be in the nanometre range, in the micrometre range or in the millimetre range. It is possible here, for example, for the coating to have an average layer thickness of 0.01 to 0.3 mm, of 0.03 to 0.1 mm, of 0.02 to 0.15 mm, of 0.03 to 0.1 mm, of 0.04 to 0.09 mm, of 0.04 to 0.08 mm, of 0.05 to 0.07 mm, of 0.09 to 0.5 mm, of 0.09 to 1 mm or of 0.5 to 2 mm. The coating may also be a droplet (for instance a droplet at at least one terminal end of the hair or a droplet at a kinked or kinkable site in the hair) or any other shape.

The hair can be anchored by any means suitable for the purpose. In one embodiment of the present invention, implantable hair in the implantable section does not have a thickening corresponding to a maximum circumference of more than twice the average circumference of the hair. This enables the use of a cut-off hair that has not been prepared further (for example cut-off human hair) for implantation, which is coated in an implantable section suitable for the purpose with the composition containing at least one silk protein. This can give a hair implant coated with the composition containing at least one silk protein without artificially simulated hair root and/or simulated hair follicle and/or simulated hair bulb, and hence without specific anchoring.

As used herein, the average circumference of the hair should be understood as the average circumference considered over the entire length of the hair. Typically, a hair, especially a cut-off hair, is largely cylindrical, such that the average circumference exists virtually over the entire length.

In one embodiment of the present invention, implantable hair in the implantable section has an artificial thickening which is formed by the composition (containing at least one silk protein), wherein the thickening corresponds to a maximum circumference around the hair of at least twice the average circumference of the hair.

Thus, it is possible to adjust the circumference of the artificial thickening which is formed from the composition according to the field of use. For example, it may have a maximum circumference of 2.1 times to 50 times, of 2.5 times to 30 times, of 3 times to 20 times or of 4 times to 10 times the average circumference of the hair. Such a structure enables firm anchoring in the skin. In this case, the composition can optionally be degraded over time after implantation in the skin. For improvement of the anchoring of the implant, it is optionally possible here to form an artificial form of the hair root and/or the hair follicle and/or a hair bulb. The artificial form here may optionally vary from the natural form, and optionally have additional structures that can improve anchoring. In this way, it is possible to obtain a hair implant coated with the composition containing at least one silk protein, with an artificially simulated hair root and/or simulated hair follicle and/or simulated hair bulb and hence with specific anchoring.

In order to improve the anchoring of the implantable hair, the implantable hair may have an artificial anchoring structure in the implantable section.

It is optionally possible for the composition containing at least one silk protein to be used for forming an anchoring in the hair implant in order to enable or improve the securing of the hair implant in the skin. The anchoring may have different forms. It may take the form of a hair root and/or of a hair follicle and/or of a hair bulb, without simulating the anatomical interior of a hair root and/or of a hair follicle and/or of a hair bulb. It may be similar to the form of a hair root and/or of a hair follicle and/or of a hair bulb.

But it may also be at distinct variance from the form of a hair root and/or of a hair follicle and/or of a hair bulb. An anchoring structure may optionally be selected from the group consisting of the form of a cone, a frustocone, a thickened cylinder, a mushroom, a droplet, an ellipsoid, a cuboid (i.e. a cube or an elongated rectangle), a spiral form, a screw form, a ring form, a sphere, a honeycomb structure, a partial wrapping of the hair with a fibre or a tape, and a combination of two or more of these, and may optionally have appendages that project from the hair structure and/or the anchoring structure (including barbs).

In one embodiment, an anchoring structure is selected from the group consisting of the form of a cone, a frustocone, a thickened cylinder, a mushroom, a droplet, an ellipsoid, a cuboid (i.e. a cube or an elongated rectangle), a spiral form, a screw form, a ring form, a sphere, and a combination of two or more of these, and may optionally have appendages that project from the hair structure (optionally and/or the anchoring structure) (including barbs).

Specific examples of anchoring structures are described herein and shown by way of example in FIGS. 2 to 27. It will be understood here that the structures are appreciated three-dimensionally by the person skilled in the art. Preference is given to assuming a largely symmetrical three-dimensional form.

The person skilled in the art is aware of the abovementioned basic forms. It will additionally be appreciated that an anchoring structure overall is preferably somewhat broader than the thickness of the hair (or the sum total of the hairs combined collectively by an anchoring structure) in the coated section. In a preferred embodiment, the anchoring structure, without optional protruding appendages, has a maximum diameter (in the plane of the diameter of the coated hair or hair bundle) of 1.1 to 20 times, especially 1.2 to 10 times, especially 1.5 to 5 times, the thickness of the average diameter of the hair (or of the sum total of the hairs combined collectively by an anchoring structure) in the coated section. This enables a good balance between efficient and gentle implantation of the hair on the one hand and good and comparatively firm anchoring in the skin on the other hand.

Wrapping of the hair with a fibre or tape can be effected in any manner suitable for the purpose. A fibre may have a (largely) round or (largely) oval cross section or (largely) square (including cuboidal) cross section. A fibre may be composed of (largely) homogeneous material or else optionally also be composed of thinner fibres (e.g. be braided, twisted or in the form of parallel fibres). A tape has a two-dimensional geometry. A tape may be composed of (largely) homogeneous material or else optionally also be composed of thinner fibres and/or tapes. A fibre or tape in this connection may be understood broadly as a composition according to the present invention comprising or consisting thereof. The thickness of a fibre or tape may be chosen freely. A thick fibre or thick tape having a diameter equal to that of the hair or exceeding the thickness of the hair may optionally be used. This enables rapid wrapping. A thin fibre or thin tape having a diameter less than that of the hair may optionally be used. This enables (largely) seamless wrapping and enables the production of (particularly) well-defined forms. The wrapping may be linear (i.e. (largely) parallel, in spiral form) winding, mesh-like winding, braid-like winding (cross-wound and/or transverse-wound) or a combination of various windings. FIG. 27 shows, by way of example, a linear (i.e. (largely) parallel, in spiral form) winding, with the outer sheath (11) constituting a spiral winding and, in this example, with additional inclusion of a further anchoring structure (9). The winding may optionally be effected directly on the hair or else on a different structure already adjoining the hair, especially a structure composed of a composition according to the present invention and containing at least one silk protein. Wrapping can be effected in any manner suitable for the purpose. For example, the hair (or bundle of hair) can be rotated (preferably about its (largely) rotationally symmetric axis) and one or more fibres and/or one or more tapes can be wound onto it as it rotates. Alternatively, it is also possible for the hair (or bundle of hair) to remain immobile and for the wrapping to be achieved by revolving material (e.g. a revolving spindle). Optionally, the wrapped material may be bonded to the hair (for example its keratin structure). It is optionally possible to bond various sections of one or more fibres and/or of one or more tapes to one another. This may prevent unwanted unwinding. For example, a spiral wrapping may have fibres bonded to at least one end, such that unwinding is prevented. Methods suitable for the purpose are described further down.

The coating containing at least one silk protein can interact under some circumstances with body fluids, for instance water, at the implanted site in the body. This can increase the volume of the coating, which can enable or enhance the anchoring. For instance, in some embodiments of the present invention, the coating can absorb tissue fluid after implantation and hence optionally swell. This can improve the anchoring of the hair in the skin.

The configurations according to the invention as described herein can give a hair implant coated with the composition containing at least one silk protein, with anchoring, the form of which varies from the natural hair root and/or the natural follicle and/or the natural hair bulb. The anchoring may have a wide variety of different forms as set out above. The thickening may thus have various forms and also incisions or protuberances. Barbs and spiral forms are also additionally or alternatively possible. Such an artificial securing form may optionally be configured such that it can optionally spread out after the implantation of the implantable hair and enable securing or improvement of the securing of the hair implant. At the same time, the composition containing at least one silk protein, in this context too, may serve for protection from bacteria and other microbes and/or to reduce or prevent an immune reaction of the body. The anchoring of the implantable hair may also comprise one or more loops in the hair.

An artificial anchoring structure may also be formed from the composition containing at least one silk protein.

It may also be formed additionally or exclusively from one or more other materials and then coated with the composition containing at least one silk protein. Such other materials may, for instance, be plastics, preferably plastics that are biologically well tolerated, for instance silicone, (methyl) methacrylate, polyvinylchloride (PVC), polylactic acid or other tolerated plastic materials or mixtures thereof. It is also possible to use inert metals, for instance gold, platinum or titanium and alloys thereof. An artificial anchoring structure may optionally also be coated with graphene. An artificial anchoring structure has preferably been coated with a composition containing a silk protein.

In one embodiment of the present invention, the implantable hair is notched in the implantable section. This too enables anchoring. For instance, the growing of dermal tissue and/or connective tissue into the indentations of the structure of the implantable hair is enabled. It is also optionally possible for dermal tissue and/or connective tissue to grow into a looped structure in the implantable hair.

In one embodiment of the present invention, the implantable hair has a hair follicle or a hair root or a hair bulb coated with the composition in the implantable section. This may enable complete and lasting growth of the hair (endogenous hair or exogenous hair). It is thus possible for the implanted hair to be fed by the skin and under some circumstances even to show lengthwise growth. In this connection too, the coating of the hair root and/or simulated hair follicle and/or a simulated hair bulb with the composition containing at least one silk protein can protect the hair implant from bacteria and microbes and/or reduce or prevent an immune reaction.

In one embodiment of the present invention, the implantable hairs are configured such that they can be implanted singly. They then therefore have a coating structure usually containing a single hair. In an alternative embodiment of the present invention, two or more implantable hairs are encompassed in a coating or anchoring structure in their implantable sections. In this way, it is possible to obtain a hair implant coated with the composition containing at least one silk protein, with an artificially simulated hair root and/or simulated hair follicle and/or simulated hair bulb and hence with specific anchoring, which may contain one hair or multiple hairs.

The coating with the composition containing at least one silk protein may be homogeneous. Alternatively, the coating may also be inhomogeneous, i.e. have different strength and/or biological stability in different regions. In one embodiment of the present invention, the coating has zones having different strength and/or biological stability. In one embodiment of the present invention, the coating has higher strength and/or biological stability in the interior close to the hair than on the outside.

This can enable faster degradation of the outer layer after the implantation of the implantable hair, and hence good enabling of ingrowing of the dermal tissue and/or connective tissue, while the inner portion protects the hair for a long period and enables prolonged holding in the skin. In this way, it is possible to obtain a hair implant coated with the composition containing at least one silk protein with a true hair root and/or true hair follicle and/or true hair bulb that has been taken from a person's head hair or body hair and can be reinserted in the same person or a different person.

When the implantable hair is inserted in a different person, this is a transplantation of exogenous hair. The immune system typically identifies it and acts against it as a foreign body. However, the immune reaction can be reduced or prevented by coating with the composition containing at least one silk protein. This can prevent a rejection reaction. The acceptance of the exogenous hair by the recipient and their tissue and immune system can be improved.

In one embodiment of the present invention, the coating of the implantable hair comprising the at least one silk protein is configured such that, after implantation, the silk protein or a degradation product is transported onto the hair outside the body and/or the scalp, where it cares/nourishes the hair and/or the skin (for instance the scalp).

The composition containing at least one silk protein may optionally also be used for coating of other materials, especially silicone or plastics, on which the hair or multiple hairs can be secured for implantation (or transplantation).

In a preferred embodiment, the implantable hair was coated with the composition by means of a method of three-dimensional (3D) shaping, especially 3D printing.

3D shaping in connection with the present invention may also be regarded as 3D printing. 3D shaping methods (e.g. 3D printing) suitable for the application (also addition) and/or removal (also subtraction) of compositions containing at least one silk protein are known to the person skilled in the art. A 3D shaping method in connection with the present invention may accordingly be a subtractive method (i.e. removal of already coated material) or an additive method (i.e. addition of material) or a combination of the two.

In a preferred embodiment, a 3D shaping method in the context of the present invention comprises the use of a pulse of light, preferably in the ultraviolet (UV) spectral region, more preferably from a femtosecond laser, especially in the UV region, as described by way of example in Sidhu et al. (“The processing and heterostructuring of silk with light”, Nature Materials, 2017, DOI: 10.1038/NMAT4942, also published as: Nature Materials, 16:938-945). Such a method enables very precise removal of material (e.g. silk protein), very precise cuts into material (e.g. silk protein) and the sticking of one material (e.g. silk protein) onto another material, for instance hair material (e.g. keratin) and of identical or similar materials to one another (e.g. a silk protein onto identical or different silk protein). This enables subtractive and additive 3D shaping methods and combinations thereof.

A femtosecond laser emits pulses of light, the duration of which is in the region of a few femtoseconds or less (femtosecond: 10⁻¹⁵ s). The laser light propagated at the speed of light, in vacuum and in air and similar gases at atmospheric pressure, covers only a distance of about 0.3 μm within one femtosecond, which corresponds to about one hundredth of the diameter of a human hair. One technical advantage of a femtosecond laser can therefore be the exact and residue-free removal of very small amounts of material without significant heat transfer (hence the principle of the precision scalpel). The material can also be converted by a femtosecond laser directly to plasma which barely interacts thermally with the carrier material.

A further technical advantage of the use of a femtosecond laser in connection with the present invention may be that a composition containing at least one silk protein can be exposed to a pulse of light (optionally even a weaker pulse of light, therefore lower in energy), preferably ultraviolet (UV) light. In this way, the composition can alter its nature for a very brief moment. It is possible here for a solid or pasty composition to become liquid, for instance, for a brief moment and then to become solid or pasty again. If the composition, during such a pulse of light with a femtosecond laser, is contacted with a different material (e.g. with a hair or a section thereof), it can stick to that material. In this way, it is possible to achieve preferably lasting adhesion of the composition to the hair or a section thereof.

The method according to the invention may also comprise a process step of forming an anchoring of the composition containing at least one silk protein in the section of the hair to be implanted. In this case, the hair can first be contacted with the composition containing at least one silk protein. The composition can then be exposed to a relatively weak pulse of light, for instance from a femtosecond laser, at the contact site with the hair. This can achieve the effect that the composition adheres to the hair at the contact site with the hair. It is thus possible to form a layer with the composition containing at least one silk protein.

This method can also be employed in an equivalent and analogous manner in order to combine further layers of the composition containing at least one silk protein with the composition. The layers thus formed may also have different strengths.

A hair or a section thereof may be contacted partly or fully with the composition containing at least one silk protein. Thereafter, the composition can be exposed to a pulse of light (a somewhat weaker pulse of light, therefore lower in energy) from the femtosecond laser. As a result, the composition can adhere to the contact site with the hair. By this method, the hair can be partly or else fully bonded to the composition, by means of which it can be partly or else fully ensheathed by the composition. This can contribute to strengthening of the hair, extension of the lifetime of the hair, protection thereof from environmental influences and/or improvement in the styling of the hair.

In one embodiment of the present invention, the section of the hair to be implanted can be contacted with one or more fibres and/or one or more tapes containing or consisting of the composition of the present invention. This may, as set out above, optionally also be accomplished by means of wrapping, as set out more specifically above. The wrapping of the hair can optionally also be conducted while the hair is in direct or indirect contact with a liquid or pasty composition.

It is optionally possible to bond a wrapped material to the hair (for example its keratin structure). This can be effected in any desired manner, by way of example by means of a pulse of light, especially in the UV region, as described herein. This can more preferably be achieved by means of a femtosecond laser, as described herein. A pulse of light, especially in the UV region, especially a femtosecond laser, as described herein, additionally permits reworking of a wrapping. This may, for example, also be a stabilization of the wrapping by (partial) bonding of various sections of the fibres/tapes.

In one embodiment of the present invention, the section of the hair to be implanted may be contacted with the liquid or pasty composition containing at least one silk protein. The section to be implanted may optionally, for instance, be immersed into such a composition, or coated or sprayed therewith. Further options are described herein. A portion of the composition may remain here (for example in droplet form) on the section of the hair to be implanted. Thereafter, the liquid or pasty composition may dry and/or cure on the section of the hair to be implanted. Curing herein should be understood in its broadest sense, such that strength increases. As a result, the composition preferably forms an anchoring firm enough to be implantable. In this case, the level of hardness is preferably sufficient to achieve a desired half-life in the surrounding body tissue, especially the skin. However, the composition preferably remains elastic. Elasticity of the anchoring structure may be advantageous in order to be perceived to a lesser degree, if at all, after implantation, and to cause less of a pressure mark, if any, and/or less pain, if any. In order to accelerate curing, the section to be implanted, after being contacted with the liquid or pasty composition, may also dry under air, or else alternatively by variation of the environment, for example with the aid of ventilation or by changing the temperature. Thereafter, the desired structure of the hair anchoring composed of the cured composition may be cut off and/or removed by a laser, for example a femtosecond laser.

In a preferred embodiment, the production of the implantable hair in the context of the present invention comprises the at least partial removal of silk protein. In a preferred embodiment, 3D shaping in connection with the present invention comprises the at least partial removal of silk protein by means of a pulse of light, preferably in the ultraviolet (UV) spectral region. It is possible here to exploit the fact that silk proteins and optionally also other components of this composition can absorb in the UV region. In a preferred embodiment, 3D shaping in connection with the present invention comprises the at least partial removal of silk protein by means of plasma ablation. In a preferred embodiment, 3D shaping in the context of the present invention comprises the at least partial removal of silk protein by means of a femtosecond laser, especially in the ultraviolet (UV) spectral region. A method based on plasma ablation by means of a femtosecond laser, especially in the ultraviolet (UV) spectral region, is known in principle to the person skilled in the art (Sidhu et al., Nature Materials, 2017, DOI: 10.1038/NMAT4942, also published as: Nature Materials, 16:938-945). The removal is preferably effected by means of a brief pulse of light of duration <100 ps (picoseconds) or <10 ps or <1 ps or <100 fs (femtoseconds) or <10 fs. In the case of a femtosecond laser, it is therefore possible by means of stronger pulses of light, especially in the UV light region, to remove ultrasmall amounts of material of the liquid or pasty or solid composition containing at least one silk protein.

By means of the precise possible applications of a femtosecond laser, it is possible to form a wide variety of forms of anchoring on the hair. It is possible, for example, to form honeycomb structures that may contain cavities. Further examples are shown by way of example in the figures. Resultant cavities and/or edges may be contacted with liquid or pasty composition containing at least one silk protein. The composition can then be exposed to a pulse of light from the femtosecond laser in order to improve or enable adhesion of the composition to the honeycomb structure.

In a preferred embodiment, the production of the implantable hair comprises the at least partial (for instance thermal) melting of the composition and/or of the at least one silk protein, especially by means of a femtosecond laser, especially in the ultraviolet (UV) spectral region.

What is meant by melting in the broadest sense is that the composition is at least partly liquefied or at least the viscosity is reduced. Melting is melting by means of a, usually brief, usually local, increase in temperature. The increase in temperature is preferably brought about by the incidence of photons. Melting is preferably melting merely of a portion of the composition and/or of a silk protein by means of a pulse of light, especially a pulse of UV light.

As an alternative to a pulse of UV light, it is also possible to use a high radiation intensity of a longer-wave light (with accordingly lower-energy photons). It is possible here to utilize multiphoton effects (e.g. 2-photon and/or 3-photon effects), as described in Sidhu et al. (Nature Materials, 2017, DOI: 10.1038/NMAT4942, also published as: Nature Materials, 16:938-945).

After the brief melting, the structure preferably cures again. The melting is preferably effected by means of a brief pulse of light of duration <100 ps or <10 ps or <1 ps or <100 fs or <10 fs. Preference is given to melting and curing a portion of the composition and/or of the silk protein for a very brief period of time, i.e. in the region of <100 ns (nanoseconds) or <10 ns or <1 ns or <100 ps or <10 ps or <1 ps or <100 fs or <10 fs. This can bring about shaping of the 3D surface structure in the nano- and/or micrometre range in that ultrasmall droplets of the at least partly molten material adhere in a defined manner to the hair or an already cured structure.

In a preferred embodiment, the 3D shaping method comprises the partial removal of silk protein that has been applied to the hair, especially by means of plasma ablation, especially by means of a femtosecond laser, especially in the ultraviolet (UV) spectral region.

Methods that can be used in connection with the present invention can also be used for simultaneous processing of multiple hairs (for instance even hundreds or thousands) and coating thereof with the composition containing at least one silk protein. Coating methods may be automated.

For instance, it is also possible to use the 3D shaping methods described for simultaneous processing of multiple hairs (for instance even hundreds or thousands) and coating thereof with the composition containing at least one silk protein. For this purpose too, it is possible to use a pulse of light (e.g. a pulse of UV light), for instance a femtosecond laser. For this purpose, several hairs may be contacted by their sections to be implanted with the composition containing at least one silk protein (e.g. by immersion into a liquid or pasty composition, for instance a thin layer in a petri dish). Thereafter, it is possible to achieve a firmer consistency of the composition (for example by ventilation, variation in temperature or variation in light, for example incidence of UV light). As soon as the composition is firm enough to keep its shape and adheres well to the hair, the hairs with their hair anchorings may be removed with the aid of a femtosecond laser. It is possible here to use short pulses of light in order to shape the anchoring form for each hair and then to part the hair together with the anchoring formed from the composition. Alternatively or additionally, the hair anchoring can then be formed even further with the femtosecond laser to give a specific anchoring form, for example in order to achieve a conical form, to form appendages, a ring form and many others. A femtosecond laser may be used to form a wide variety of different anchoring forms, and these forms may also be combined with one another, for example including a honeycomb form, ring form with appendages etc. Structure examples are described herein and are also shown by way of example in FIGS. 2 to 27.

In a preferred embodiment, the implantable hair has been at least partly coated with a solid or pasty composition having a 3D surface structure in the nano- or micrometre range that differs from the largely cylindrical form (i.e. a form similar to the hair, and preferably also a droplet form). Possible shapes are described herein and by way of example in FIGS. 2 to 27.

The implantable hair according to the present invention may be produced in any manner suitable for the purpose. Processes as described herein constitute preferred embodiments.

A further aspect of the present invention also relates to a method of producing implantable hair according to the present invention, wherein the method comprises the following steps:
(i) providing:
   (A) hair that is not on the human or animal body, and
   (B) a composition containing at least one silk protein; and
(ii) contacting at least an implantable section of the hair with the composition.

Corresponding examples of processes have already been set out above; further examples are set out further hereinafter, and in the examples section.

It will be understood that all definitions and embodiments given in connection with the implantable hair according to the present invention are correspondingly also applicable to the method of producing such an implantable hair.

In a preferred embodiment, the present invention relates to a method of producing implantable hair according to the present invention, wherein the method comprises the following steps:
(i) providing:
   (A) hair that is not on the human or animal body, and
   (B) a liquid or pasty or solid (preferably liquid or pasty) composition containing at least one silk protein;
(ii) contacting at least an implantable section of the hair with the liquid or pasty or solid composition; and
(iii) optionally drying the implantable hair obtained from step (ii).

Steps (ii) and (iii) can optionally be repeated twice or more than twice, which can enable a higher layer thickness and/or the achievement of any desired forms of coating.

The present invention also relates to implantable hair obtainable or obtained by a method as described herein.

The hair used in step (i) of providing the starting materials may be any hair. It may be, for instance, a human hair, an animal hair or a synthetic hair. Corresponding embodiments are elucidated in detail in connection with the implantable hair.

The liquid or pasty or solid composition containing at least one silk protein which is used in step (i) of providing the starting materials may be any liquid or pasty or solid (preferably liquid or pasty) composition.

The liquid or pasty or solid (preferably liquid or pasty) composition containing at least one silk protein which is used for coating may contain a solvent or solvent mixture. The at least one silk protein is preferably soluble in a solvent or solvent mixture, or the at least one silk protein forms a colloid or hydrogel in a solvent.

A solvent may be a cosmetically compatible solvent. A cosmetically compatible solvent may be selected, for example, from the group consisting of water, a cosmetically compatible buffer, a hydroalcoholic mixture containing water and up to 5% by volume of ethanol, dimethyl sulfoxide (DMSO), and combinations of two or more of these. A cosmetically compatible solvent may optionally remain wholly or partly on the implantable hair. It can alternatively be evaporated, for instance by drying.

A solvent may also be an organic solvent. This preferably has at least a degree of polarity that enables solubility of or formation of a colloid by the at least one silk protein. For example, an organic solvent may be selected from one or more alcohols (e.g. ethanol, methanol, propanol and/or butanol), acetone, acetonitrile. At least if the organic solvent is not cosmetically compatible in the concentration used, the organic solvent is preferably evaporable, i.e. can be wholly or partly removed by evaporation.

The liquid or pasty or solid (preferably liquid or pasty) composition used for coating may, as well as a solvent, optionally also contain one or more preservatives, one or more antibacterial (for instance bactericidal and/or bacteriostatic) agents, one or more antifungicidal agents, one or more antiviral agents, one or more detergents, one or more chelators, one or more haemostatic agents, one or more vitamins, one or more bioactive substances that promote hair growth or perfusion, one or more local anaesthetics, one or more thickeners, one or more hormones, one or more acid regulators, or a combination of two or more thereof.

The terms “liquid” and “pasty” should be understood as used in general linguistic usage. A liquid composition at room temperature (20° C.) under standard pressure (1013 mbar) preferably has a viscosity of less than 5000 mPa·s (for example determined with a capillary viscometer). A pasty composition at room temperature (20° C.) under standard pressure (1013 mbar) preferably has a viscosity of at least 5000 mPa·s (for example determined with a capillary viscometer).

The term “solid” should be understood in its broadest sense as having a greater solidity than a pasty composition, i.e. not free-flowing at room temperature (20° C.) under standard pressure (1013 mbar). For instance, a soap may be liquid or pasty, or else in the form of a solid bar of soap (including hand soap). The term “solid” may also include waxy.

In one embodiment, the silk protein may have a solid or viscous pasty (waxy) structure. This can achieve good technical properties by compression through an injection needle and reduce or prevent damage (for instance to silk protein) on injection and hence improve the protective function of the silk protein.

Such a solid or viscous pasty (waxy) structure can optionally be achieved by subjecting a liquid or pasty composition to thermal drying and/or air drying until it becomes solid or viscous and pasty (waxy). This can optionally also be effected with the aid of additional chemical substances.

The contacting step (ii) can be effected in any manner suitable for the purpose. In one embodiment of the present invention, the contacting step (ii) comprises the immersing of the hair or a section thereof into the liquid or pasty or solid (preferably liquid or pasty) composition. In a further embodiment of the present invention, the contacting step (ii) comprises the coating of the hair or a section thereof with the liquid or pasty or solid composition. In a further embodiment of the present invention, the contacting step (ii) comprises the spraying of the hair or a section thereof with the liquid or pasty or solid composition. It will be recognized that combinations of different method steps of contacting may optionally also be combined with one another.

In one embodiment of the present invention, the contacting step (ii) comprises the combining of a hair (or bundle of hair) with a (largely) solid composition containing one or more silk proteins. In one embodiment of the present invention, the contacting step (ii) comprises the insertion of a hair (or bundle of hair) into a prefabricated (largely) solid anchoring structure. In one embodiment of the present invention, the contacting step (ii) comprises the wrapping of a hair (or bundle of hair) with one or more fibres and/or one or more tapes.

It will be understood that multiple method steps of contacting may also be combined with one another. This can be effected simultaneously and/or successively in time. For instance, by way of example, wrapping or insertion into a (largely) solid anchoring structure can also be effected in a liquid or pasty composition.

The contacting step (ii) can be conducted at any desired temperature at which at least the primary structure of the at least one silk protein used is preserved and not denatured, and therefore no amino acid residues of the at least one silk protein break down by pyrolysis. In one embodiment of the present invention, step (ii) is conducted at temperatures in the range from −40 to 250° C., −20 to 200° C., −10 to 180° C., −5 to 150° C., 0 to 150° C., from 2 to 120° C., from 3 to 100° C., from 4 to 80° C., from 4 to 60° C., from 4 to 50° C., from 4 to 40° C., from 5 to 30° C., from 10 to 25° C. or from 15 to 25° C. In one embodiment of the present invention, step (ii) is conducted at room temperature in the range from 15 to 25° C. or from 18 to 22° C., especially (about) 20° C.

If another manner of contacting at least an implantable section of the hair with the liquid or pasty or solid composition is possible in step (ii), for instance by means of vapour deposition on the hair or a section thereof (at elevated temperature and/or under reduced pressure), such a procedure is also encompassed by the present invention. It is also optionally possible to subject the coating with silk protein to vapour deposition in turn.

The contacting step (ii) can be effected at any desired pressure. The step is preferably conducted at ambient pressure, for instance in the range from 960 to 1100 mbar, from 980 to 1050 mbar or from 990 to 1040 mbar.

Step (ii) of contacting at least an implantable section of the hair with the liquid or pasty or solid composition may be the contacting of a terminal end of the hair with the liquid or pasty or solid composition. In that case, it is possible, for instance, to dip a terminal end of a single hair into such a composition or to dip a terminal end of a combined bunch of multiple hairs into the composition. It is also possible, for instance, to coat a terminal end of a single hair with a composition or to coat a terminal end of a combined bunch of multiple hairs with the composition. It is also possible, for instance, to spray a terminal end of a single hair with a composition, or it is possible to spray a terminal end of a combined bunch of multiple hairs with the composition.

Step (ii) of contacting at least an implantable section of the hair with the liquid or pasty or solid composition may also be the contacting of both terminal ends of the hair with the liquid or pasty or solid composition. In that case, the hair can, for instance, be pulled over a rod such that both ends are positioned on one side, for instance hang down, and both ends are dipped into or coated or sprayed with the composition. This is analogously also possible with a bundle of multiple hairs.

In a preferred embodiment, the method of the present invention gives a 3D surface structure in the nano- or micrometre range that differs from a largely cylindrical form.

Difference from a largely cylindrical form is regularly particularly advantageous for technical reasons since the adhesion of an implanted hair in the scalp is better. The implanted hair can be anchored more firmly and durably.

Step (ii) of contacting at least an implantable section of the hair with the liquid or pasty or solid composition may optionally also be effected by means of 3D shaping (e.g. 3D printing).

In a preferred embodiment, the contacting step (ii) comprises the sticking of the composition to the hair or a section thereof by means of a 3D shaping method, giving a 3D surface structure in the nano- or micrometre range that differs from the largely cylindrical form of the hair (and preferably also a droplet form).

In a preferred embodiment, the contacting step (ii) comprises the sticking of the composition to the hair or a section thereof by means of the partial melting of the composition and/or of the at least one silk protein, especially by means of a femtosecond laser, especially in the ultraviolet (UV) spectral region.

In a preferred embodiment, the method comprises the further step of partly removing silk protein that has been applied to the hair.

In a preferred embodiment, this is effected by means of plasma ablation. In a preferred embodiment, this is effected by means of a femtosecond laser, especially in the ultraviolet (UV) spectral region.

These methods are set out further above.

In a preferred embodiment, the method comprises the following steps:
(i) providing:
   (A) hair that is not on the human or animal body, and
   (B) a largely solid or pasty composition containing at least one silk protein that may optionally constitute one or more fibres or an at least partly dried composition according to the present invention;
(ii) contacting at least an implantable section of the hair with the composition, or keeping them in contact, where steps (i) and (ii) may optionally also take place simultaneously,
(iii) sticking the composition to the hair or a section thereof by means of at least partial melting of the composition containing at least one silk protein, preferably by means of a pulse of light, especially by means of a femtosecond laser, especially in the ultraviolet (UV) spectral region.

Step (ii) of contacting at least an implantable section of the hair with the liquid or pasty or solid composition may also be the contacting of a kink or kinkable section of the hair with the liquid or pasty or solid composition. In that case, the hair may, for instance, be pulled over a rod such that both ends are positioned on one side, for instance hang down, and the tip present at the bend of the rod is dipped into the composition or is coated or sprayed therewith. This is analogously also possible with a bundle of multiple hairs.

Alternatively, the hair in step (ii) may also be dipped fully into the liquid or pasty or solid composition containing at least one silk protein or be completely coated or sprayed therewith. This is also possible with multiple hairs simultaneously. It is optionally possible to use the hair obtained from step (ii) for implantation. In this case, for instance, the hair can be coated at the site of implantation of the hair or in the spatial proximity thereof.

Optionally, the hair obtained from step (ii) may also be subjected to an additional step (iii) of drying. This can extend the storability of the implantable hair. Drying can be effected under any desired conditions. The conditions here are preferably chosen such that neither the hair nor the at least one silk protein is broken down by pyrolysis. The drying may take place at room temperature (15 to 25° C., preferably about 20° C.) or at elevated temperature (for instance in the range from above 20° C. to 150° C. or from 30° C. to 100° C. or from 40° C. to 70° C.). The drying may take place at standard pressure or under reduced pressure (for example under vacuum). The drying may also be a spray-drying operation, in which case the coated hair from step (ii) is deep-frozen or shock-frozen (preferably at temperatures of <−10° C., <−20° C., <−60° C. or, for instance, −196° C.) and then the solvent is evaporated under reduced pressure. The drying may optionally also take place in a stream of air. The drying may optionally also be accelerated with a hairdryer.

The resultant implantable hair may be stored under any suitable conditions, for instance at ambient temperature or in a refrigerator. It can optionally be packaged. It may, for example, be packaged in a sterile manner. It may, for example, be packaged under reduced pressure or under protective gas atmosphere. It may be packaged in transparent or light-protective packing. It may be packed and/or stored, for example, in a liquid or pasty composition containing at least one silk protein.

It will be understood that a silk protein, hair and/or the coated hair according to the present invention can optionally be stored at any temperature suitable for the purpose, for instance at temperatures in the range from −200 to 250° C., −100 to 250° C., −80 to 200° C., −50 to 180° C., −10 to 150° C., 0 to 150° C., from 2 to 120° C., from 3 to 100° C., from 4 to 80° C., from 4 to 60° C., from 4 to 50° C., from 4 to 40° C., from 5 to 30° C., from 10 to 25° C. or from 15 to 25° C. Preferably, if it is not stored at ambient temperature, it is brought to a temperature close to ambient temperature prior to implantation, for instance from 5 to 30° C., from 10 to 25° C. or from 15 to 25° C. As in many materials, spider silk fibres undergo a glass transition. The glass transition temperature may depend on the humidity. Water can function here as plasticizer.

The present invention also relates to a liquid or pasty or solid composition containing at least one silk protein which is suitable for coating of the hair or an implantable section thereof.

The present invention also relates to a concentrate or powder for production of a liquid or pasty or solid (preferably liquid or pasty) composition containing at least one silk protein which is suitable for coating of the hair or an implantable section thereof.

A packing may optionally contain use instructions that teach, among other things, how the implantable hair can be implanted into the skin.

The present invention therefore also relates to a kit (of parts) comprising:
(A) implantable hair according to the present invention; and
(B) use instructions for implantation.

The present invention also relates to a kit comprising:
(A) hair;
(B) at least one vessel containing a liquid or pasty or solid (preferably liquid or pasty) composition containing at least one silk protein which is suitable for coating of the hair or an implantable section thereof;
(C) optionally use instructions for coating of the hair with the composition; and
(D) optionally use instructions for implantation.

The present invention also relates to a kit comprising:
(A) hair;
(B) at least one vessel containing a concentrate or powder for production of a liquid or pasty or solid (preferably liquid or pasty) composition containing at least one silk protein which is suitable for coating of the hair or an implantable section thereof;
(C) optionally use instructions for coating of the hair with the composition;
(D) optionally use instructions for implantation; and
(E) optionally at least one solvent for production of the liquid or pasty composition from the concentrate or powder.

As described, the implantable hair can be used for implantation into human or animal skin.

Therefore, a further aspect of the present invention relates to the (cosmetic) use of implantable hair for implanting into human or animal skin, especially the human scalp.

A further aspect of the present invention thus encompasses a (cosmetic) method of implanting implantable hair according to the present invention into human or animal skin, especially the human scalp, comprising the following steps:
(i) providing hair according to the present invention;
(ii) introducing the hair into the human or animal skin, especially human scalp, preferably by means of one or more needles.

It will be understood that all definitions and embodiments made in connection with the implantable hair and the production process therefor according to the present invention are also correspondingly applicable to the use of or a method of using such an implantable hair.

In one embodiment, the implantation is effected into the scalp. In another embodiment, the implantation is effected into one or more other regions of the face or body. For instance, implantation can be effected in the region of one or both eyebrows, in the region of one or both eyelashes, in the moustache region, in the chin region of the beard, in the cheek region of the beard, in the region of one or both sideburns, in the region of the armpits, in the pubic region, in the region of one or both arms, in the chest region, in the region of one or both legs. Appropriate hair may be selected according to the area of use.

The implantation can be effected for any desired purpose. The aim will typically be to increase hair coverage in one or more areas of the human or animal skin.

In one embodiment, the implantation is preferably effected largely symmetrically in both halves of the body. In the event of an accident and/or scarring, however, it may also be appropriate to subject one side of the body to greater or even exclusive implantation. The implantation can be effected for establishment or reestablishment of hair coverage of the head. For emphasis of male body features too, implantation of hair as body hair may also be of interest, for example in the case of a gender transition. It is also possible to implant longer eyelashes. A particular shape of a beard may also be achieved by implantation of implantable hair according to the present invention. The implantation of the implantable hair according to the invention may also be used for the reconstruction of eyebrows, eyelash transplantation after Alopecia mechanica, Alopecia areata, beard replacement, for instance after correction of a harelip, and/or (optionally scarring traumatic) alopecia.

Optionally, in addition to an inventive implantation of hair, it is also possible to simulate hair coverage and/or to conceal partial or complete lack of hair in some other way. For instance, it is also possible to trace eyebrows by application of makeup/paint. Another option is tattooing of the visual appearance of short hair and/or the colour of the scalp.

The implanted implantable hair according to the present invention may optionally grow permanently in the recipient's skin. It may optionally also migrate to the epidermis in the course of skin renewal and ultimately fall out. The duration for which it remains in the skin can be defined by the anchoring as described above. For instance, it is possible to simulate the natural life cycle of hair. The half-life can be chosen collectively with the recipient.

For instance, the half-life of the implanted hair can be chosen. The falling-out of hair can be increased and accelerated by tactile and mechanical effects, for instance combing, washing of the corresponding part of the head or body, wind, hair drying, friction by clothing etc.

Implantation can in principle be conducted by any known method. The person skilled in the art is aware of numerous methods in principle.

The composition containing at least one silk protein may also be used to coat hair present on the surface of the head or body of a human or animal, for instance in order to protect the hair from environmental effects or obtain improved properties for styling.

This is particularly true when the hair present on the surface of the head or body of a human or animal is an implanted hair according to the present invention.

A further aspect of the invention relates to a method of cosmetic haircare, wherein the hair is selected from the group consisting of
(a) implanted implantable hair according to the present invention on the head or body,
(b) hair extensions,
(c) stickable (e.g., self-adhesive) (stuck-on) eyelashes,
(d) eyelash extensions,
(e) wigs, and
(f) toupees,
wherein the method includes the step of applying a liquid or pasty or solid (preferably liquid or pasty) composition containing at least one silk protein to the hair.

It will be understood that all definitions and embodiments made in connection with the implantable hair, the production method therefor according to the present invention and the use of such an implantable hair are also applicable correspondingly to the method of cosmetic care of the implanted implantable hair.

The hair may be coated directly or else optionally already have been coated by one or more other layers. The composition containing at least one silk protein may optionally also coat other layers that have been applied to the hair beforehand. The composition containing at least one silk protein may also coat the hair before, during or after the bleaching and also before, during or after the colouring of the hair. The coating may remain on the hair and/or migrate into the hair. The hair may be coated completely or partially. This can optionally improve the hair structure, the optical properties of the hair and the styling. It is also possible to increase protection from environmental influences and the lifetime of the hair.

One embodiment of the invention relates to a method of cosmetic care of the implanted implantable hair according to the present invention on the head or body, comprising the step of applying a liquid or pasty composition containing at least one silk protein to the hair.

The step of applying the liquid or pasty or solid (preferably liquid or pasty) composition can be effected in any manner suitable for the purpose. The composition may be present, for example, in a haircare, beardcare or personal-care oil, in a haircare, beardcare or personal-care lotion, in a haircare, beardcare or personal-care cream, in a haircare, beardcare or personal-care powder, in a shampoo, in a rinse, in a shower gel, in a body milk or another cosmetic product for hair- or skincare (for example in mascara).

The liquid or pasty or solid (preferably liquid or pasty) composition for care may optionally be packed together with use instructions for hair care and take the form of a kit.

A further aspect of the invention relates to a method of cosmetic haircare, wherein the method includes the step of applying a liquid or pasty or solid composition containing at least one silk protein to the hair, and wherein the hair:
(a′) has been lightened and/or coloured prior to the step of applying the composition containing at least one silk protein,
(b′) is lightened and/or coloured during the step of applying the composition containing at least one silk protein, or
(c′) is lightened and/or coloured after the step of applying the composition containing at least one silk protein,
wherein the hair is preferably a hair according to the present invention on the head or body, wherein the hair is preferably as defined above.

The person skilled in the art will appreciate that the term “lightened” may be understood in its broadest sense as imparting a lighter shade to the hair. Lightenting of the hair can be conducted on one hair (e.g. implanted implantable hair according to the present invention) on the body or on an implantable hair according to the present invention outside the body. The person skilled in the art is aware of various methods of lightening. For example, bleaching is possible (for example by means of a peroxide-containing solution (for example an H₂O₂-containing aqueous solution)), or it is possible to use an osmosis bath, or a combination thereof. It is possible here to open up the scales of the hair in order to remove the colour pigments therefrom. The duration and intensity and also the temperature and pH will be chosen by the person skilled in the art depending on the original hair colour and the target colour for the hair.

The person skilled in the art will appreciate that the term “coloured” may be understood in its broadest sense as colouring of a hair. Colouring of the hair may be conducted on one hair (e.g. implanted implantable hair according to the present invention) on the body or on an implantable hair according to the present invention outside the body. In order to colour the hair, colour pigments are typically supplied (optionally again). This can be effected in any desired manner, for example by coating, spraying and/or by means of an osmosis bath. The duration and intensity and also the temperature and pH will be chosen by the person skilled in the art depending on the original colour of the hair (optionally lightened beforehand) and the target colour for the hair. For colouring, it is possible to use any dyes suitable for the purpose, for example natural and/or synthetic colour pigments. When the hair is being coloured lighter, it is possible to remove natural pigments from the hair and simultaneously to deposit synthetic pigments. This procedure is employed preferably when the hair is not to change its original colour but is merely to lighten in colour. Dark blonde hair may become light blonde, for example. Dark brown hair may become caramel-coloured, for example. This operation constitutes a lower level of stress on the hair; nevertheless, the hair can also be damaged in this procedure.

A hair in connection with a method of cosmetic haircare may be endogenous hair, exogenous hair or synthetic hair. Further embodiments are set out herein. Hair may, for example, be temple hair that is being treated. The hair is preferably (implanted or non-implanted) implantable hair according to the present invention.

In order to colour (including to dye) (implantable) hair, it may be advantageous that it is first lightened and/or subjected to an osmosis bath. Before the colouring of the hair, the hair may optionally be subjected to drying, ideally gentle drying until it is moist or dry. When the hair is being coloured lighter, it is possible to remove natural pigments from the hair and simultaneously or subsequently to deposit synthetic pigments. This may be the procedure when the hair is not to change its original colour but is merely to lighten in colour. Dark blonde hair may become light blonde, for example. Dark brown hair may become caramel-coloured, for example. This operation constitutes a lower level of stress on the hair; nevertheless, the hair can also be damaged in such a procedure. In the blonding, lightening and colouring of the hair alike, the hair structure can be damaged. The thinner the hair in the starting situation and/or the more bleaching and/or colouring is necessary, the greater the damage to the hair structure or scale layer of the hair can be.

In order to care for the hair, it can be contacted with a liquid and/or pasty and/or solid (preferably liquid or pasty) composition containing at least one silk protein before, during and/or after the lightening and/or before, during and/or after the colouring. Such a composition is preferably defined as described herein. This can be effected in any desired manner, for example by dipping, coating or spraying the hair. Embodiments are described above. By means of an osmosis bath, the hair can absorb the composition containing at least one silk protein particularly efficiently and form an inert protection. In this way, it is possible to strengthen the structure, to protect the hair from environmental influences, and to improve the styling of the hair. It is also possible to maintain the hair colour for longer. The life cycle of the hair can be extended.

The figures, claims and examples that follow describe further embodiments of the invention that further illustrate the invention.

### DESCRIPTION OF THE FIGURES

FIG. 1 shows a schematic of the hair implantation of one or more hairs (2) into the human scalp (1). It is additionally shown that the implantation of hair can also be effected in the region of the eyebrow (3), of the eyelashes (4), of the moustache (5) or of the beard (6) (for example the chin or cheek area of the beard or sideburns). It is likewise possible to conduct the implantation of hair at other places on the body as well. For better illustration, the transplantation of hair is shown viewed through a magnifying glass (7), with the coating of the implantable section (here by way of example of the implantable terminal end) of a hair (2) in FIG. 1 represented by way of example as a simulation of a hair root or a hair follicle or a hair bulb (8) comprising silk protein (e.g. spider silk and/or caterpillar silk).

FIGS. 2 to 27 show examples of structures that can serve for anchoring of the implantable hair (2) according to the invention. For instance, it is possible to use a structure in the form of a simulation of a hair root or a hair follicle or a hair bulb (8) and comprising silk protein (e.g. spider silk and/or caterpillar silk) that optionally comprises an additional anchor structure (9). It is also possible for a structure that can serve for anchoring of the implantable hair (2) according to the invention to have one or more incisions (10) and hence to achieve improved anchoring. As can be seen in the figures, an anchoring may optionally contain an additional anchor structure (9) composed of a composition comprising silk protein (e.g. spider silk and/or caterpillar silk) or coated therewith, which has the function of barbs, a spiral form as in the case of a screw or protuberances (9) in order to enable or to improve the securing of the hair implant. It is also possible for the coating, the optional anchoring and/or the optional structures of the anchoring to have different strengths. In addition, the anchoring (9) may be annular, which can improve the ingrowing of the surrounding tissue. It is also possible for the anchoring (9) optionally to have one or more appendages with a composition containing silk protein (e.g. spider silk and/or caterpillar silk) that are connected to the anchoring by a kind of bridge or any other form of connection. The anchoring may also have a structure, for example in honeycomb form, that may have a higher hardness than the surrounding anchoring. The implanted hair (2) may optionally have a complete or partial sheath (11), preferably in the implantable section thereof, consisting of a composition comprising silk protein (e.g. spider silk and/or caterpillar silk). It is also possible for an implantable hair (2) to comprise a natural/real hair root (12) and/or hair follicle and/or hair bulb that may be surrounded by a coating (13) with a composition comprising silk protein (e.g. spider silk and/or caterpillar silk).

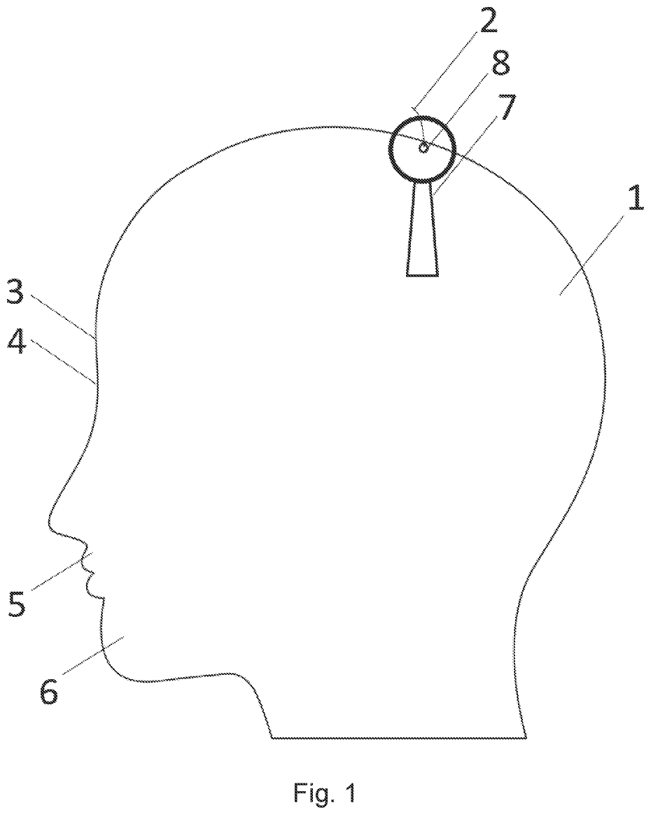

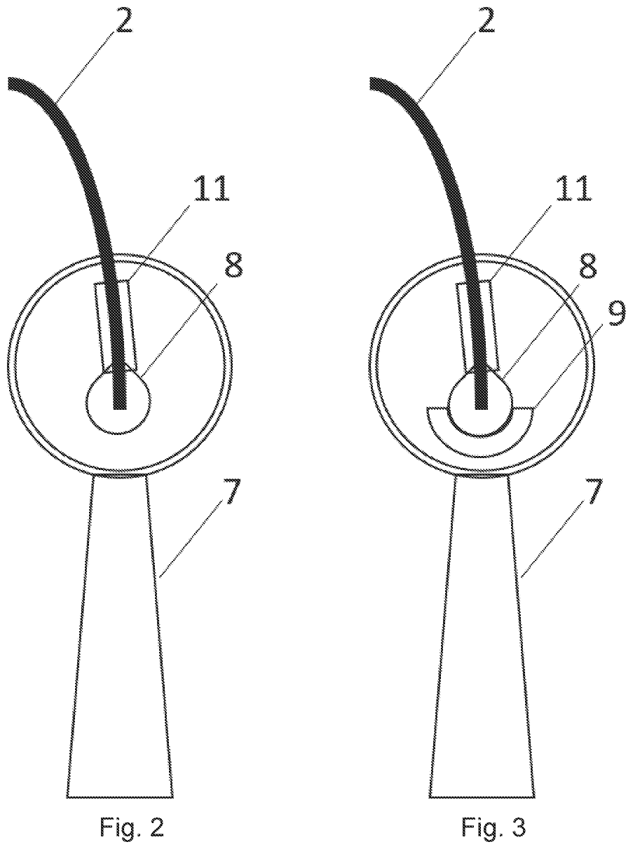

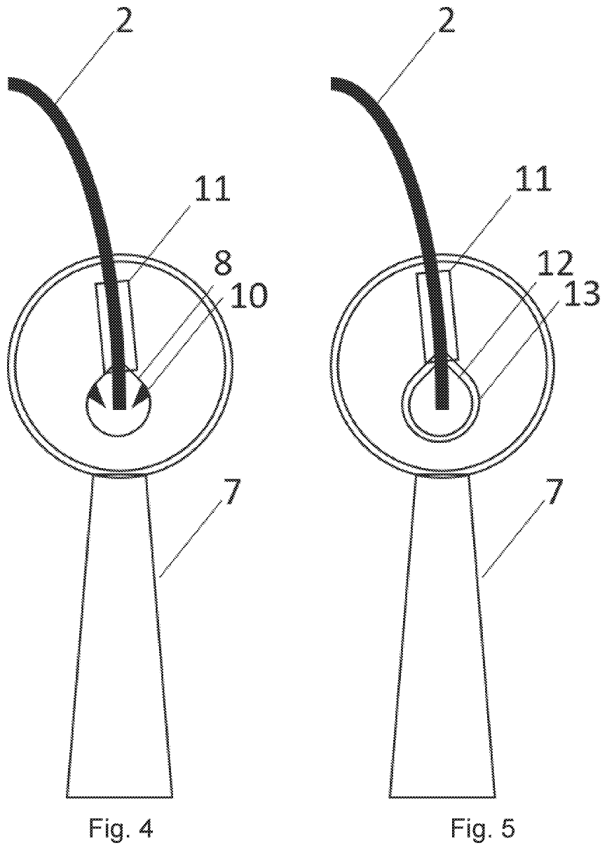

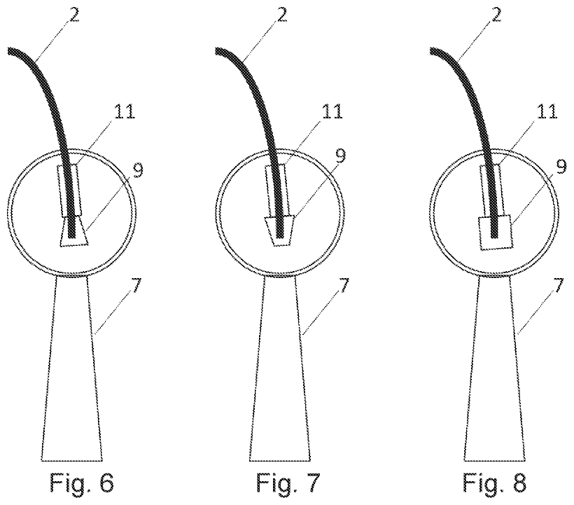

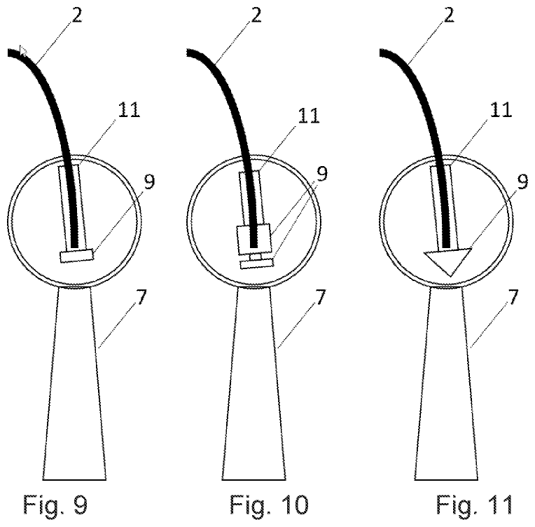

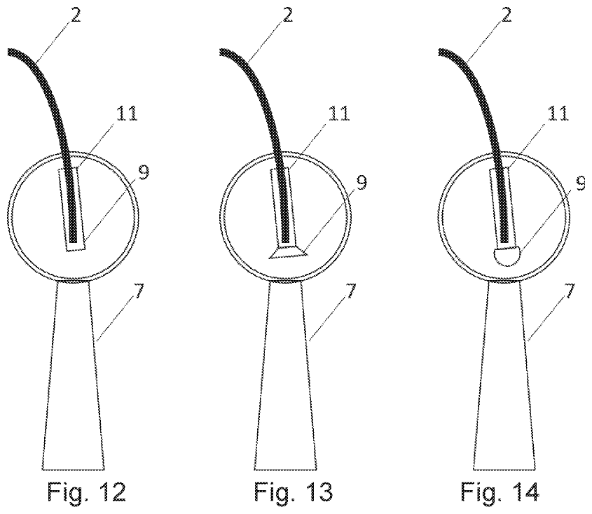

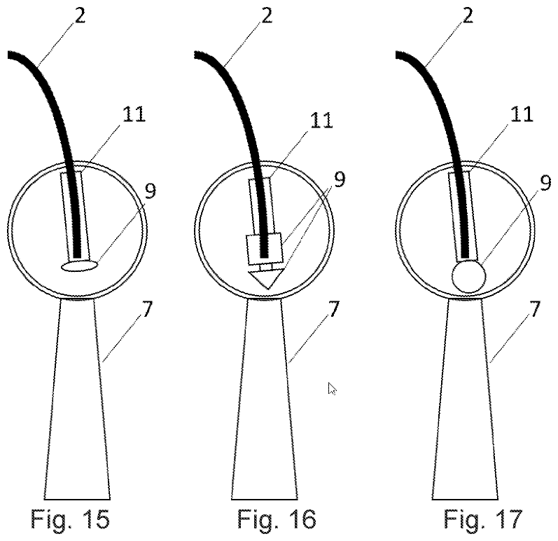

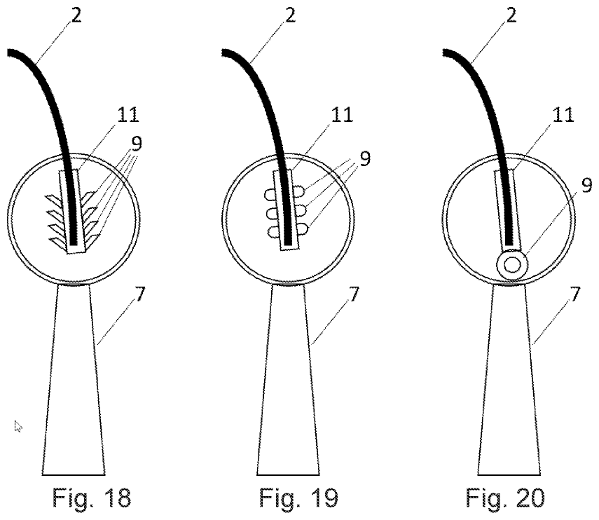

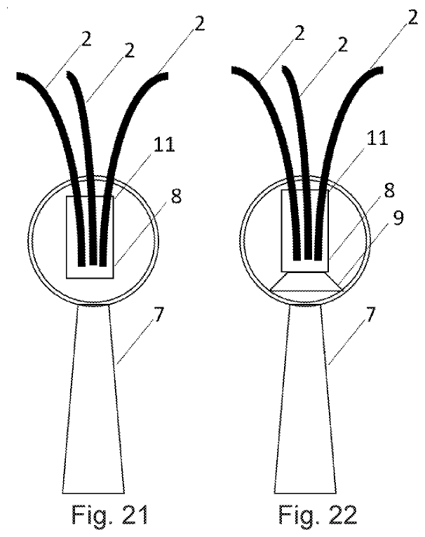

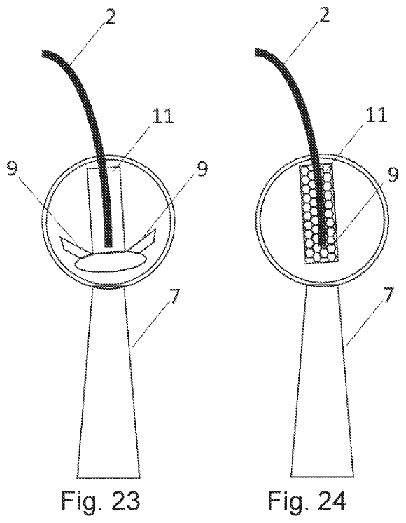

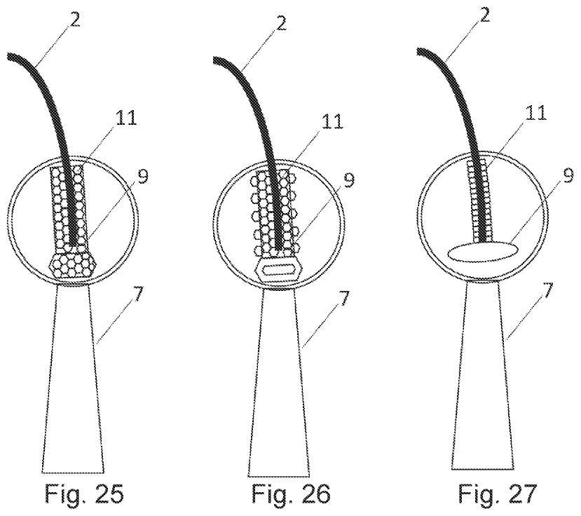

### EXAMPLE

The spider silk may be obtained in various ways. By way of example, a silk protein as described in WO 2007/025719 and WO 2006/008163 is used, i.e. a fibroin-3 and/or -4 from the European garden spider (Araneus diadematus). This is also referred to as “Araneus diadematus fibroin 3” (ADF-3) or “Araneus diadematus fibroin 4” (ADF-4) or else as (“Major Ampullate Spidroin II” or “Spidroin 2”). The ADF-3 and/or ADF-4 spider silk protein which is described here by way of example can be obtained commercially. It was produced as described in WO 2006 008163. Hence, in a bacterial fermentation process, bacteria were produced by Escherichia coli (also called E. coli or colibacterium). Spider silk protein-encoding DNA from the European garden spider (Araneus diadematus) was inserted into the E. coli bacteria by gene transfer. This created genetically modified E. coli bacteria that express spider silk protein by fermenting beets or sugarcane, for example.

The implantable hair for implantation comes mainly from India. It is customary for Hindu pilgrims to sacrifice their hair after taking a vow or when starting a new stage in their life. This takes place predominantly at Indian temples, and therefore the hair is also referred to as temple hair. In order to prepare the hair for the European market, for example, they are sorted according to their properties, for example according to the length, bundled, bleached and coloured.

For the implantation of the implantable hair, however, it is also possible to cut off endogenous hair. In order to assure good quality, it should if at all possible be unbleached and uncoloured. For coating of the implantable hair, it is sorted and fixed by bundling in narrow strips. At the terminal end of the hair, the hairs are cut off with a very sharp blade in order that they all end in the same place. This ensures that every single hair is coated with silk. This is followed by the coating with silk by immersing the implantable section of the hair into liquid or pasty composition or rubbing on the solid composition. For better distribution of the silk, the bundle of hair is rotated while being immersed into the liquid or pasty composition.

Thereafter, the hair is subjected to drying. For acceleration of drying, it is subjected to gentle air ventilation. For formation of further silk coatings, the hair, after the silk layer has dried, is immersed again into the liquid or pasty composition by the same principle.

These process steps are repeated until the desired thickness of the protective layer around the implantable hair and the anchoring has been attained. In order additionally to establish a mushroom-shaped anchoring, for example, the end of the hair to be implanted, starting from a certain process repetition, is immersed into the liquid or pasty composition only up to the place where the mushroom cap-shaped broadening is to form.

Prior to implantation, the scalp is first locally anaesthetized. This is done with a plunger filled with a local anaesthetic, for instance by means of the “Comfort-in” injection device (Mika Medical Co. 93, Noksansaneopjung-ro, Gangseo-gu, Busan, Republic of Korea). This plunger has, at the lower end, a nozzle having an opening of about 0.15 mm. As soon as the trigger is pressed, the plunger is in contact with the skin for about 0.3 seconds, and within this short space of time applies the anaesthetic at high pressure beneath the skin, which leaves it undamaged. This anaesthetizes all layers of the skin.

In order to implant the implantable hair into the skin, especially the scalp, a specific device known for this purpose in the art is used, which is similar to a propelling pencil. For this purpose, it is possible, for instance, to use conventional CII hollow needles (punches) from Cole Instruments Inc. (1070 Powers Place, Alpharetta GA 30009 USA) with appropriate commercially available additional equipment. Such a device comprises an outer hollow needle with which the skin is penetrated and an inner hollow needle into which the implantation hair is introduced and in which it is held by a clamping mechanism. At the hold point is an advance mechanism (likewise similar to a propelling pencil), with the aid of which the implantation hair is pushed about 1 mm out of the tip of the inner hollow needle at its terminal end. The electrical trigger mechanism for advancing the inner hollow needle and releasing the hold is controlled by actuating a foot pedal.

The internal diameter of the inner hollow needle may be about 0.05 to 0.6 mm broader than the anchoring of the hair implant. The inner diameter of the outer hollow needle is only broader to the extent required for the inner hollow needle to be able to slide through. The outer diameter of the outer hollow needle is about 0.2 to 0.9 mm wide.

The implantation device, in the direction of hair growth, is leant against the skin at an angle corresponding to the natural exit angle of the hair, at about 45 degrees. The outer hollow needle is introduced into the skin. Then the trigger pedal is pressed, as a result of which the inner hollow needle is pushed forward automatically through the outer hollow needle. As soon as the inner hollow needle reaches the tip of the outer hollow needle, the hair is pushed forward by the advance mechanism. As soon as the hair has advanced as described, the clamp mechanism is released automatically and the hair is released. Then the implantation device is removed again from the skin and prepared for the implantation of the next hair.

An alternative method is the implantation of the hair with a device as already used in the transplantation of endogenous hair. For this purpose, for example, it is possible to use the “OKT Implanter” manufactured by Cole Instruments Inc. (1070 Powers Place, Alpharetta GA 30009 USA), the needle of which may have a diameter of 0.6 to 1.14 mm. In the transplantation of endogenous hair, the hair removed, together with its hair root, is placed into a needle having an incision on one side or a passage opening. Then the hair is pushed into the scalp and implanted. Also in the case of implantation of implantable hair according to the invention with a coating containing at least one silk protein and optionally having an anchoring structure, the hair together with the anchoring can be placed into the needle of the OKT Implanter, pushed into the skin and implanted. It is possible here for the hair to hang out at the needle opening, which makes it possible to implant long hair has well.

Depending on the number of implantable hairs to be implanted, a treatment may take a few hours to a few days.

1-25. (canceled)

26. An implantable hair comprising at least one implantable section, wherein the at least one implantable section is coated with a composition comprising at least one silk protein, wherein the composition comprising at least one silk protein comes into direct contact with the implantable hair.

27. The implantable hair according to claim 26, wherein the at least one silk protein has at least 90% amino acid sequence homology to a spider silk protein or an insect silk protein.

28. The implantable hair according to claim 26, wherein the at least one silk protein is:
(a) selected from the group consisting of fibroin, sericin, spidroin 1 and spidroin 2; or
(b) comprises at least two sequence sections each having one of the following sequences SEQ ID NO: 1 to 6:
(SEQ ID NO: 1)
GPGXX,
(SEQ ID NO: 2)
GVPGX,
(SEQ ID NO: 3)
GSGXX,
(SEQ ID NO: 4)
GGYXX,
(SEQ ID NO: 5)
PQQXX,
(SEQ ID NO: 6)
GYGXX,
wherein X is a natural amino acid.

29. The implantable hair according to claim 26, wherein the at least one implantable section is an implantable terminal end of the hair, an implantable kink that is not located at a terminal end of the hair, or a kinkable portion that is not located at a terminal end of the hair.

30. The implantable hair according to claim 26, wherein the implantable hair is a human hair.

31. The implantable hair according to claim 26, wherein the implantable hair comprises a human hair, wherein the human hair was removed from its follicle, root, and bulb.

32. The implantable hair according to claim 26, wherein the implantable hair comprises a synthetic hair or an animal hair.

33. The implantable hair according to claim 26, wherein the implantable hair, over its entire length, has an average diameter of 0.02 to 0.15 mm.

34. The implantable hair according to claim 26, wherein the implantable hair comprises at least one of the following features:
(1) an implantable section with a circumference of no more than twice the average circumference of the hair;
(2) an implantable section, wherein the coating composition thickens the implantable section to a maximum circumference of at least twice the average circumference of the uncoated section of the hair;
(3) an implantable section comprising an artificial anchoring structure, wherein the structure is, optionally, selected from the group consisting of the form of a cone, a frustocone, a thickened cylinder, a mushroom-shaped, a droplet form, an ellipsoid, a cuboid, a spiral form, a screw form, a ring form, a sphere, a honeycomb structure, a partial wrapping of the hair with a fiber or a tape, and a combination of two or more of the foregoing option, and may optionally, comprise appendages that project from the hair structure and/or the anchoring structure;
(4) an implantable section comprising at least one notch;
(5) an implantable section comprising a hair follicle, a hair root, and/or a hair bulb coated with the composition;
(6) an implantable section comprising zones, wherein the coating imparts different strength and/or biological stability in comparison to uncoated sections of the implantable hair, optionally wherein the different strength and/or biological stability is higher in the interior of the hair than it is on the outside of the hair; and
(7) the composition comprising at least one silk protein or degradation product thereof which, after implantation, is transported onto the hair outside the body and/or the scalp, where it nourishes the hair and/or the skin.

35. The implantable hair according to claim 26, wherein the composition comprising at least one silk protein is coated onto the hair using 3D shaping, optionally 3D printing.

36. The implantable hair according to claim 35, wherein the composition comprising at least one silk protein is at least partially melted when coated on the implantable section, optionally wherein a femtosecond laser is used to melt the at least one silk protein.

37. The implantable hair according to claim 26, wherein the silk protein was partially removed, optionally by using plasma ablation or a femtosecond laser.

38. The implantable hair according to claim 26, wherein the composition comprising at least one silk protein is pasty or solid, and wherein the implantable hair has a 3D surface structure in the nano- or micrometer range that differs from a largely cylindrical form.

39. A method of producing the implantable hair of claim 26, wherein the method comprises the following steps:
(i) providing:
(A) a hair that is not on a human or animal body, and
(B) a composition comprising at least one silk protein; and
(ii) contacting the at least an implantable section of the hair with the composition comprising at least one silk protein.

40. The method according to claim 39, wherein the composition is a liquid or pasty or a solid composition, and the method further comprises the step of:
(iii) drying the implantable hair obtained from step (ii).

41. The method according to either of claim 39, wherein the composition comprising at least one silk protein is pasty or liquid, and wherein the contacting step (ii) comprises dipping the hair or a section thereof into the liquid or pasty composition comprising at least one silk protein.

42. The method according to claim 39, wherein the contacting step (ii) comprises coating or spraying of the hair or a section thereof with the composition comprising at least one silk protein.

43. The method according to claim 39, wherein the contacting step (ii) comprises sticking the composition to the hair or a section thereof by means of a 3D shaping method, thereby resulting in a 3D surface structure in the nano- or micrometer range that differs from a largely cylindrical form.

44. The method according to claim 39, wherein the contacting step (ii) comprises sticking the composition to the hair or a section thereof by partially melting the composition comprising at least one silk protein.

45. The method according to claim 39, wherein the method further comprises a step of partly removing silk protein that has been applied to the hair.

46. The method according to claim 39, wherein the method comprises the following steps:
(i) providing:
(A) hair that is not on the human or animal body, and
(B) a largely solid or pasty composition comprising at least one silk protein that may optionally constitute one or more fibers or an at least partly dried composition comprising at least one silk protein;
(ii) contacting or maintaining contact of at least an implantable section of the hair with the composition, where steps (i) and (ii) may optionally also take place simultaneously,
(iii) sticking the composition to the hair or a section thereof by partially melting the composition, optionally by using a pulse of light, optionally a pulse of light from a femtosecond laser, preferably forming a 3D surface structure in the nano- or micrometer range that differs from a cylindrical form.

47. A cosmetic use of the implantable hair of claim 26 for implanting into human or animal skin.

48. A cosmetic method of implanting the implantable hair of claim 26 into human or animal skin comprising implanting the implantable hair of claim 26 into the human or animal skin by means of a needle.

49. A method of cosmetic haircare, comprising a step of applying a liquid or pasty or solid composition comprising at least on silk protein to hair, wherein the hair is selected from the group consisting of:
(a) implanted implantable hair according to claim 26 on the head or body;
(b) hair extensions;
(c) stickable eyelashes;
(d) eyelash extensions;
(e) wigs; and
(f) toupees,
wherein the method comprises the step of applying a liquid or pasty or solid composition containing at least one silk protein to the hair.

50. A method of cosmetic haircare, comprising a step of applying a liquid or pasty or solid composition comprising at least one silk protein to the hair, wherein the hair:
(a) has been lightened and/or colored prior to the step of applying the composition containing at least one silk protein;
(b) is lightened and/or coloured during the step of applying the composition containing at least one silk protein; or
(c) is lightened and/or coloured after the step of applying the composition containing at least one silk protein.
